SEQUENCE LISTING (5 sequences)

SEQ ID NO: 1 - DNA, 12 bp, Artificial Sequence
  upstream open reading frame
accatgggttga

SEQ ID NO: 2 - DNA, 20 bp, Artificial Sequence
  primer
ggtgtctaagggcgaagagc

SEQ ID NO: 3 - DNA, 20 bp, Artificial Sequence
  primer
gctggtagccaggatgtcga

SEQ ID NO: 4 - DNA, 20 bp, Artificial Sequence
  primer
gtaacccgttgaaccccatt

SEQ ID NO: 5 - DNA, 20 bp, Artificial Sequence
  primer
ccatccaatcggtagtagcg

(19) United States
(12) Patent Application Publication
(10) Pub. No.: US 2023/0159948 A9
(43) Pub. Date: May 25, 2023

(54) ENGINEERED INCOHERENT FEED FORWARD LOOP AND USES THEREOF

(71) Applicant: Massachusetts Institute of Technology, Cambridge, MA (US)

(72) Inventors: Ron Weiss, Newton, MA (US); Ross D. Jones, Cambridge, MA (US); Breanna E. DiAndreth, Cambridge, MA (US); Domitilla Del Vecchio, Cambridge, MA (US); Yili Qian, Madison, WI (US)

(73) Assignee: Massachusetts Institute of Technology, Cambridge, MA (US)

(21) Appl. No.: 17/153,276

(22) Filed: Jan. 20, 2021

Related U.S. Application Data
(60) Provisional application No. 62/992,829, filed on Mar. 20, 2020.

(51) Int. Cl. C12N 15/85; C12N 15/113; C12N 9/22

(52) U.S. Cl. CPC C12N 15/85; C12N 15/113; C12N 9/22; C12N 2310/20; C12N 2830/50

(57) ABSTRACT

The present disclosure, at least in part, relates to an engineered incoherent feed forward loop (iFFL) comprising a first transcription unit encoding an endoribonuclease and a second transcription unit encoding an output molecule and an endoribonuclease target site located in the 5′ UTR of the output molecule coding sequence. The engineered iFFL, at least in part, can be used for sustained expression of an output molecule despite of transcription resource disturbance in a given environment.

### RELATED APPLICATION

This application claims the benefit under 35 U.S.C. § 119(e) of U.S. provisional application No. 62/992,829 filed Mar. 20, 2020 which is incorporated by reference herein in its entirety.

### FEDERALLY SPONSORED RESEARCH

This invention was made with Government support under Grant No. MCB1840257 awarded by the National Science Foundation. The Government has certain rights in the invention.

### BACKGROUND

In synthetic biology, methods for heterologous gene expression stabilization and disturbance rejection in mammalian cells remain limited. It is difficult to establish accurate, robust, and predictable control over heterologous gene expression using the known synthetic genetic circuits currently available.

### SUMMARY

The present disclosure, at least in part, relates to an engineered incoherent feed forward loop (iFFL). An engineered incoherent feed forward loop, as used herein, refers to a class of network structure wherein an upstream node both activates and represses a downstream node through divergent branches (FIG. 1H). In some embodiments, iFFLs are designed to provide robust perfect adaptation (RPA) of output expression to upstream disturbances, such as resource availability, off-target promoter interaction, and varying gene dosage (e.g., DNA copy number) (FIG. 1G).

In some aspects, the iFFLs described herein comprise two transcription units: (i) a first transcription unit comprising a first promoter operably linked to a nucleic acid molecule encoding an endoribonuclease; and (ii) a second transcription unit comprising a second promoter operably linked to a nucleic acid molecule encoding an output molecule, and a endoribonuclease target site located within the 5′ untranslated region (UTR) of the nucleic acid molecule encoding the output molecule, wherein the endoribonuclease is capable of cleaving the endoribonuclease target site on an RNA transcript expressed by the second transcription unit. In some embodiments, the first promoter and the second promoter are identical. In some embodiments, the first promoter and the second promoter share the same transcriptional resources.

In some embodiments, the first promoter and the second promoter are not identical. In some embodiments, the first promoter is at least 80% identical to the second promoter. An iFFL with non-identical promoters are useful, in some embodiments, to adapt the output molecule expression to the available copies of the first transcription unit and the second transcription unit, such as when there are different copy numbers of the first transcription unit and the second transcription unit.

In some embodiments, the endoribonuclease is a CRISPR-associated endoribonuclease. In some embodiments, the CRISPR-associated endoribonuclease is an endoribonuclease from the Cas6 family or the Cas 13 family. In some embodiments, the CRISPR-associated endoribonuclease is CasE, Cas6, Csy4, Cse3, PspCas13b, RanCas13b, PguCas13b, or RfxCas13d.

In some embodiments, the first transcription unit further comprises at least one upstream open reading frame (uORF) located within the 5′UTR of the nucleotide sequence encoding the endoribonuclease. Such uORF are capable of regulating or fine tuning the expression of the endoribonucleases. Placement of different numbers of uORF within the 5′UTR of the nucleotide sequence encoding the endoribonuclease results in varying level of expression of the endoribonuclease. In some embodiments, the first transcription unit comprises 1, 2, 3, 4, 5, 6, 7, 8, 9, 10, 11 or 12 uORFs. In some embodiments, the uORF comprises a nucleotide sequence of ACCATGGGTTGA (SEQ ID NO: 1).

In some embodiments, the first transcription unit and the second transcription unit are present on the same nucleic acid or on different nucleic acids. In some embodiments, the first transcription unit and the second transcription unit are present on the same vector or on different vectors.

In some embodiments, the first promoter and the second promoter are constitutive promoters, inducible promoters, or tissue specific promoters.

In some aspects, the present disclosure also provides a cell comprising the engineered incoherent feed forward loop described herein.

In some aspects, the present disclosure also provides a composition comprising the engineered incoherent feed forward loop or the cell described herein. In some embodiments, the composition further comprises a pharmaceutically acceptable carrier.

In some aspects, the present disclosure also provides a method for delivering an output molecule to a subject in need thereof, the method comprising: delivering to the subject the engineered incoherent feed forward loop, the cell, or the composition described herein.

In some aspects, the present disclosure also provides a method for delivering an output molecule to a cell in need thereof, the method comprising: contacting the cell the engineered incoherent feed forward loop, the cell, or the composition described herein.

In some aspects, the present disclosure also provides a method for maintaining expression level of an output molecule to transcriptional disturbance in a subject in need thereof, the method comprising: delivering to the subject the engineered incoherent feed forward loop, the cell, or the composition. In some embodiments, the first transcription unit and the second transcription unit are delivered on the same nucleic acid. In some embodiments, the first transcription unit and the second transcription unit are delivered on the same vector. In some embodiments, the first transcription unit and the second transcription unit are delivered on different nucleic acids. In some embodiments, the first transcription unit and the second transcription unit are delivered on the different vectors. In some embodiments, the ratio between the first transcription unit and the second transcription unit is proportional.

### BRIEF DESCRIPTION OF DRAWINGS

FIGS. 1A-1H: iFFL-based approach for decoupling modules with shared limited resources. FIG. 1A: A genetic module comprising a single constitutive transcription unit. Other competing modules place a load on the free cellular resources, affecting expression of the module of interest (resource-coupled module). The module of interest also applies a load to the resources. FIG. 1B: An incoherent feedforward loop (iFFL) device within the module of interest decouples the module's output from resource variability. An endoribonuclease (endoRNase/ERN) produced by the identical promoter as the output represses the output by binding to a specific target site in its 5′UTR and cutting the mRNA. FIG. 1C: A simplified schematic of the iFFL showing how cellular resources directly contribute to the production of both the endoRNase and the output. FIG. 1D: The expected behavior of the output of the resource-coupled and resource-decoupled modules in response to resource loading by other modules. FIG. 1E: Experimental model system of resource loading. The module of interest comprises a constitutively expressed protein (Outputs). In a competitor module, Gal4 transcriptional activators (TAs) drive expression of another protein (Output2). Different activation domains (ADs) were fused to the DNA binding domain (DBD) of Ga14. A reporter (Gal4 Marker) was titrated together with the Gal4 TAs to mark their delivery per cell. Note that the transfection marker (TX Marker) also competes for resources, but that process is omitted from schematic for simplicity. FIG. 1F: Dose-dependent effect of Gal4 TAs on Outputs. The markers indicate median expression levels from three experimental repeats. The lines represent fits of the steady-state resource competition model. The CV(RMSE) is the root-mean-square error between the model and data, normalized by the mean of the data. All data were measured by flow cytometry at 48 hours post-transfection in HEK-293FT cells. FIG. 1G: Gene expression is affected by many factors that reflect cell state. These factors include resource availability, the presence of off-target interactors, and (for ectopic systems) varying gene dosage (copy number). These factors as disturbances can be modeled to be rejected with an iFFL. FIG. 1H: iFFLs are a class of network structure wherein an upstream node both activates and represses a downstream node through divergent branches. iFFLs controllers can provide robust perfect adaptation (RPA) of output expression to upstream disturbances. Illustrated are designs previously published using transcription factors (TFs) and microRNAs (miRNAs, miRs), as well as the new design using endoRNAses. Strengths/weaknesses of each design are compared in the table to the right. Notably, the condition for non-cooperativity in TF-promoter binding hampers scalability of TF iFFLs.

FIGS. 2A-2F: Effect of resource competition between promoters and activators across cell lines. FIG. 2A: Genetic model system to study competition for resources between different combinations of promoters, Gal4 transcriptional activators (TAs), and cell lines. The specific promoters, activation domains (ADs) fused to Gal4, and cell lines are shown alongside the data in FIGS. 2B-2C. FIG. 2B: Nominal Outputs are the median expression levels of each promoter in Module 1 ({P}: Output1) in each cell line when co-transfected with Gal4-None (i.e. the Gal4 DNA binding domain), which does not load resources. FIG. 2C: Fold-changes (Fold-Δs) in the level of {P}: Output1 in response to Gal4 TAs. The Fold-Δs are computed independently for each promoter and cell line by dividing the median level of {P}: Output1 for each sample co-transfected with different Gal4 TAs by the Nominal Output. FIG. 2D: The five promoter-activator combinations in each cell line with the smallest effect or largest negative effect on the level of Output1. The plots show the mean and standard deviation of three experimental repeats (represented by the individual points). FIG. 2E: Specificity of EndoRNase cutting. Various endoRNases were tested for specificity in gene knockdown. Each endoRNase (rows) was co-transfected in HEK-293FT cells with a reporter gene expressing a fluorescent protein with a target site (XXXr or XXxR, columns) in the 5′UTR of the output mRNA. The heatmap shows expression levels normalized by a transfection marker. Brightness corresponds to log-transformed output levels measured with flow cytometry. FIG. 2F: Target Site Placement. CasE EndoRNase and miR-FF4 iFFLs were tested with target sites in either the 5′ or 3′ UTR of the output mRNA. Circuit variants were co-transfected into HEK-293FT cells with a constitutive transfection marker. Adaptation of output expression to the DNA copy number was assessed by measuring the output and transfection marker by flow cytometry. The results show that adaptation to copy number only occurs when the miRNA or endoRNase are targeted to the 5′UTR. CV(RMSE)=RMSE/mean(y). All data were measured by flow cytometry at 48 hours post-transfection in the cell lines indicated. FIGS. 2B-2C show the geometric mean of median measurements and mean of fold-changes from three experimental repeats, respectively.

FIGS. 3A-3E: Adaptation to resource perturbations of an endoRNase-based iFFL module. FIG. 3A: A schematic of the iFFL module. The expression of the output gene (y) may change due to variations in the availability of free transcriptional (TX) and translational (TL) resources (R). When the gene is regulated by an endoRNase (x), an unintended increase (decrease) in R increases (decreases) the amount of endoRNase to reduce (increase) the amount of the output by enhancing its mRNA degradation. This action compensates for the unintended increase (decrease) in the regulated gene's production rate due to variations in R. Since the same pool of TX and TL resources is also used to express the transfection marker z in a transient transfection experiment, the marker's concentration z was used as a proxy to quantify R experimentally. FIG. 3B: The steady state output level (y) of the iFFL can be written as a function of the marker level (z). The performance of an iFFL was evaluated by (i) its maximum output (Ymax) and (ii) its robustness to variation in R and therefore z, characterized by (Z50). In this model, both Ymax and Z50 are linear functions of ε, which can be used as a design parameter (see equation (2)). ε is proportional to the decay rates of the endoRNase and output mRNA and is inversely proportional to the production rate and catalytic efficiency of the endoRNase. FIG. 3C: An increase in the number of uORFs in the 5′ UTR of the endoRNase's transcript leads to a decrease in its TL initiation rate. It was modeled as an increase in the dissociation constant between the ribosome and the endoRNase's mRNA transcript (κx), which increases ε. The relationship between the number of uORFs and the fold decrease in TL initiation (i.e., parameter κx in the model) is summarized in the table using previously-published experimental data38. FIG. 3D: Sample experimental data corresponding to the theoretical plot in b. n indicates the number of uORFs in the 5′UTR of CasE. The shroud indicates the 5th to 95th percentiles of the output in each half-log-decade TX Marker bin. The thin lines mark the 25th and 75th percentiles, and the thick line marks the median Output in each bin. FIG. 3E: Comparison between experimentally measured inverse robustness metric (Z50) and maximum output (Ymax) and the relative difference in ribosome-mRNA dissociation constant (κx) for different numbers of uORFs in the 5′UTR of the endoRNase.

FIGS. 4A-4B: Robustness of the iFFL output level to resource loading by Gal4-VPR. FIG. 4A: Schematic and genetic diagram of the endoRNase-based iFFL module. 0-12 uORFs are placed in front of CasE to reduce its translation. CasE binds and cleaves to a specific target site in the 5′UTR of the output mRNA. The unregulated (UR) control is the same overall design but replaces CasE with Fluc2. The UR control has no uORFs and retains the CasE target site in the 5′UTR of the output mRNA. FIG. 4B: Comparison of UR and iFFL responses to resource loading by Gal4-VPR. The median output plots show the mean (μ)±relative error Embedded Image of medians for three experimental repeats. Relative error rather than standard deviation (a) was used to more accurately represent error on the log-scale of the y-axis59. Fold-changes (Fold-Δs were computed by dividing the median level of Output at a given concentration of Gal4-VPR by the Nominal Output level (the median level of Output at 0 ng Gal4-VPR). The Fold-Δ plots show the mean±standard deviation of Fold-Δs of individual repeats. Robustness scores (see equation (7) in the text) for each UR and iFFL variant as a function of the Nominal Output. The robustness of the iFFL decreases as ε is increased. Each box or square represents an individual experimental replicate. Direct comparison of the distributions of Output at 0 and 30 ng Gal4-VPR for UR and iFFL variants show similar Nominal Outputs, for all three experimental repeats. The lines on the histograms denote the 5th, 25th, 50th, 75th, and 95th percentiles. All data were measured by flow cytometry at 72 hours post-transfection in HEK-293FT cells. All measurements were made on cells gated positive for Output. iFFL samples with 0 or 1 uORFs are not shown because most or all of the cells in those samples did not express Output above the autofluorescence background and their median expression levels were much lower than that of any UR variants.

FIGS. 5A-5F: Robustness of the iFFL output level to resource loading across cell lines. FIG. 5A: Schematic of experiment to test the performance of the iFFL in different cell lines with different Gal4 TAs loading resources. FIG. 5B: Nominal Outputs are the median expression levels of each UR or iFFL variant (Output1) in each cell line when co-transfected with Gal4-None (i.e. the Gal4 DNA binding domain), which does not load resources. FIG. 5C: Fold-changes (Fold-Δs) in the level of Output1 in response to Gal4 TAs. The Fold-Δs are computed independently for each UR and iFFL variant and cell line by dividing the median level of Output1 for each sample co-transfected with different Gal4 TAs by the Nominal Output. FIG. 5D: Distribution of Fold-Δs for each UR and iFFL variant. Histogram bins with minimal Fold-Δs are highlighted green for UR variants and blue for iFFL variants. The average (mean) of all Fold-Δs for a given UR or iFFL variant is shown inset in each plot. FIG. 5E: Distribution of robustness scores for each UR and iFFL variant. Histogram bins with >80% robustness are highlighted green for UR variants and blue for iFFL variants. FIG. 5F: Comparison of distributions of robustness scores for all UR vs iFFL variants in each cell line. The average (mean) robustness scores for each group are shown inset in each plot. All data were measured by flow cytometry at 72 hours post-transfection in the cell lines indicated. FIGS. 5B-5C show the geometric mean of median measurements and mean of fold-changes from three experimental repeats, respectively. FIGS. 5D-5F combine data from each replicate. All measurements were made on cells gated positive for Output1 only.

FIGS. 6A-6F: Adaptation of the iFFL output level to DNA copy number variation. FIG. 6A: Genetic diagram of the iFFL and a constitutive TX Marker to report plasmid dosage delivered to each cell. The hEF1a promoter is used to drive transcription of all genes. FIG. 6B: Top row: TX Marker vs Output levels for each sample, overlaid with fits of the iFFL model. The CV(RMSE) is the root-mean-square error between the model and non-binned data, normalized by the mean of the data (log10-transformed first since the cell-to-cell variance is approximately log-normally distributed). Bottom row: histograms of the Output levels for cells within each color-coded bin (as indicated in the scatters). Data is representative and taken from the first experimental replicate. FIG. 6C: Robustness of iFFL Output levels in more finely-sampled bins, computed in reference to the fit parameter Ymax. The values were log-transformed before the calculation. Bins with robustness scores over 95% (which was defined as adapted to DNA copy number) are highlighted in shades of blue. Individual replicates are shown separately. FIG. 6D: Correlation between the range of DNA copy numbers over which an iFFL variant is adapted and Z50, which is proportional ε. The adaptation range is defined as the largest sum of the log-widths of contiguous adapted bins in a sample. FIG. 6E: Median expression over time for UR and iFFL variants (including a miRNA-based iFFL for comparison). The absolute accumulated change is the sum of the absolute values of the log2 changes in median expression between time points, summed from 12 to 120 hours. FIG. 6F: Comparison of fit parameters Ymax and Z50 over time for the iFFL variants (including a miRNA-based iFFL for comparison). The results are from populations of cells gated positive for either Output or TX Marker.

### DETAILED DESCRIPTION

The present disclosure, at least in part, relates to an engineered incoherent feed forward loop for sustained expression of an output molecule despite of transcription resource disturbance in a given environment. Such an engineered incoherent feed forward loop includes two transcription unit: (i) a first transcription unit for expression of an endoribonuclease (e.g., CRISPR-associated endoribonucleases); and (ii) a second transcription for expression of an output molecule, where a endoribonuclease target site recognizable by the endoribonuclease expressed by the first transcription unit is located within the 5′ untranslated region (UTR) of the output molecule coding sequence. The first transcription unit and the second transcription unit, in some embodiments, have identical promoters, therefore the transcription activity of the two transcriptional units are coupled. In other embodiments, the first transcription unit and the second promoter of the second transcription unit of the iFFL do not have identical promoters, but the transcription of the first transcription unit and the second transcription unit are still coupled. In some embodiments, the first promoter and the second promoter are at least 80% identical to each other. In some embodiments, the first promoter and the second promoter are not identical, but still share the same transcription resources. Various designs can be combined with the engineered incoherent feed forward loop to fine tune the expression of the endoribonucleases (e.g., upstream open reading frames (uORFs)), and thus achieving fine tuning of the output molecule expression.

### I. Engineered Incoherent Feed Forward Loop

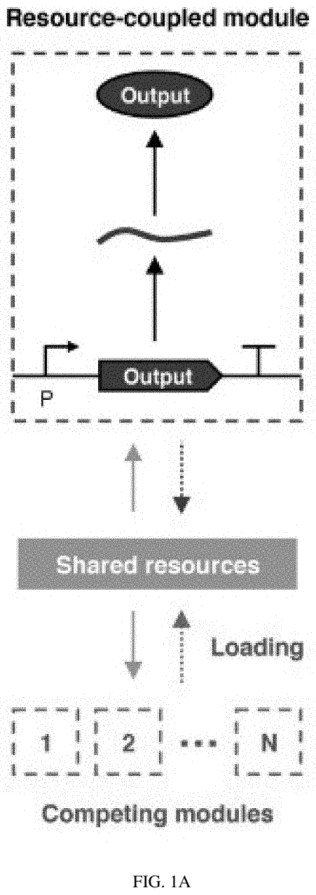

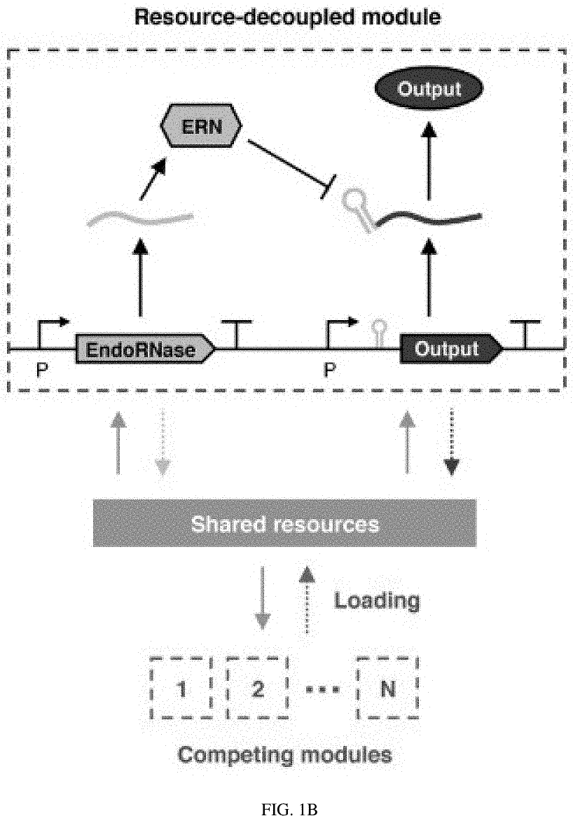

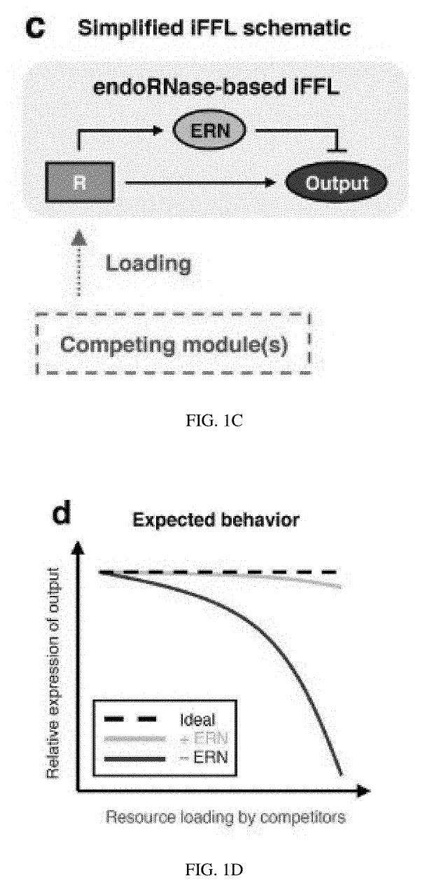

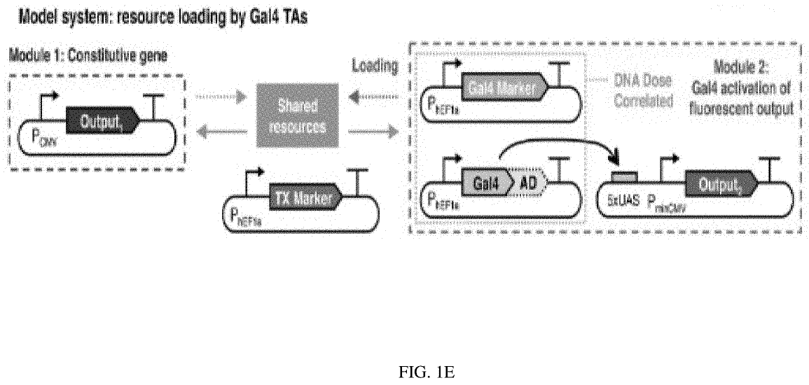

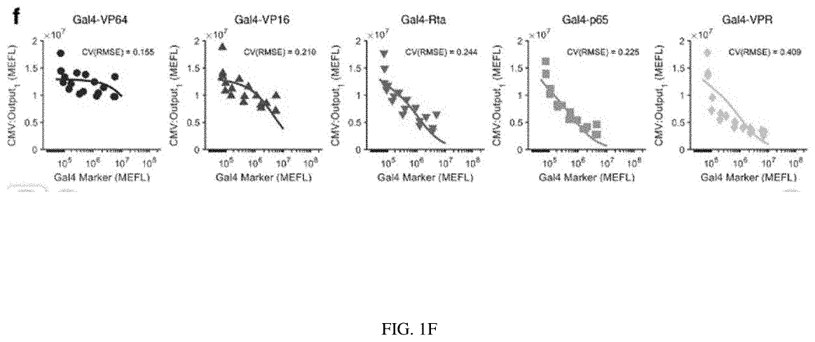

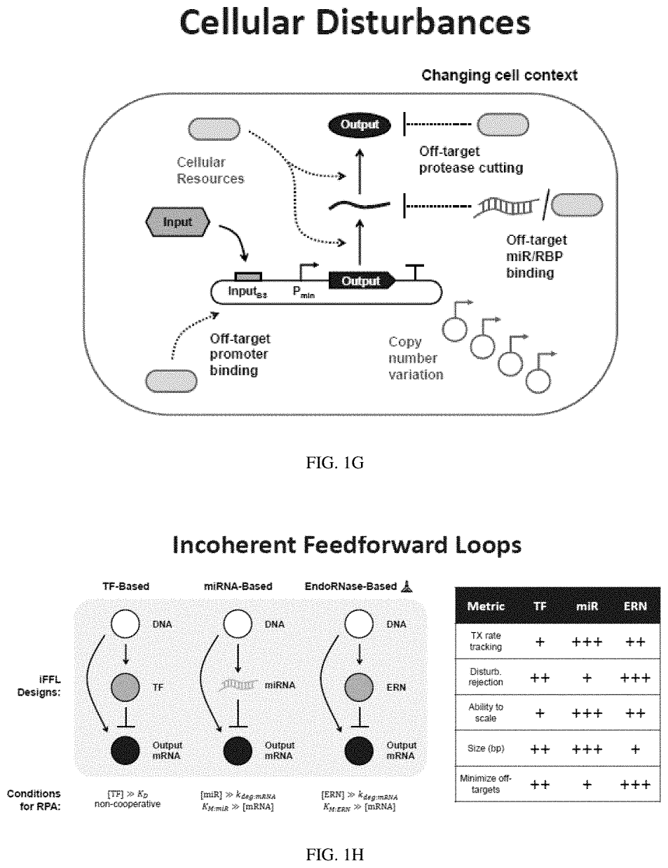

The disclosure, at least in part, provides an engineered incoherent feed forward loop (iFFLs). An engineered incoherent feed forward loop or a feed forward controller, as used herein, refers to a class of network structure wherein an upstream node both activates and represses a downstream node through divergent branches (FIG. 1H). In some embodiments, iFFLs are designed to provide robust perfect adaptation (RPA) of output expression to upstream disturbances, such as resource availability, off-target promoter interaction, and varying gene dosage (e.g., DNA copy number) (FIG. 1G). In some embodiments, the iFFLs described herein comprises two transcription units: (i) a first transcription unit comprising a first promoter operably linked to a nucleic acid molecule encoding an endoribonuclease; and (ii) a second transcription unit comprising a second promoter operably linked to a nucleic acid molecule encoding an output molecule, and a endoribonuclease target site located within the 5′ untranslated region (UTR) of the nucleic acid molecule encoding the output molecule.

In some embodiments, the endoribonuclease is capable of cleaving the endoribonuclease target site on an RNA transcript expressed by the second transcription unit. In some embodiments, the first promoter and the second promoter are identical. In some embodiments, the first promoter and the second promoter share the same transcriptional resources. In some embodiments, the first promoter and the second promoter are not identical. In some embodiments, the first promoter is at least 80% identical to the second promoter. An iFFL with non-identical promoters are useful, in some embodiments, to adapt the output molecule expression to the available copies of the first transcription unit and the second transcription unit, such as when there are different copy numbers of the first transcription unit and the second transcription unit.

A transcription unit, as used herein, refers to DNA sequences that code for a single RNA molecule, along with the sequences necessary for its transcription (e.g., promoters, and regulatory elements for transcription).

In some embodiments, the first transcription unit of the iFFL comprises a first promoter operably linked to a nucleic acid molecule encoding an endoribonuclease. An “endoribonuclease,” as used herein, refers to a nuclease that cleaves an RNA molecule in a sequence specific manner, e.g., at a target site. Sequence-specific endoribonucleases have been described in the art. For example, the Pyrococcus furiosus CRISPR-associated endoribonuclease 6 (Cas6) is found to cleave RNA molecules in a sequence-specific manner (Carte et al., Genes & Dev. 2008. 22: 3489-3496). In another example, endoribonucleases that cleave RNA molecules in a sequence-specific manner are engineered, which recognize an 8-nucleotide (nt) RNA sequence and make a single cleavage in the target (Choudhury et al., Nature Communications 3, 1147 (2012). In some embodiments, the endoribonuclease belongs to the CRISPR-associated endoribonuclease. In some embodiments, the endoribonuclease belongs to the CRISPR-associated endoribonuclease 6 (Cas6) family. Cas6 family nucleases from different bacterial species may be used. Non-limiting examples of Cas6 family nucleases include Cas6, Csy4 (also known as Cas6f), Cse3, and CasE. In some embodiments, the endoribonuclease encoded by the first transcription unit of the iFFL is CasE. In some embodiments, the endoribonuclease belongs to the CRISPR-associated endoribonuclease 13 (Cas13) family. Cas13 family nucleases from different bacterial species may be used. Non-limiting examples of Cas13 family nucleases include Cas13a, Cas13b, Cas13c, and Cas13d. In some embodiments, the Cas13 family nucleases are waCas13a, PspCas13b, RanCas13b, PguCas13b, and RfxCas13d.

In some embodiments, the expression of the endoribonuclease by the first transcription unit of the iFFL can be regulated by including additional elements into the first transcription unit. In some embodiments, the first transcription unit further comprises upstream open reading frame (uORF). An “upstream open reading frame (uORF)”, as used herein, refers to an open reading frame (ORF) within the 5′ untranslated region (5′UTR) of an mRNA. uORFs can regulate gene expression. Translation of the uORF typically inhibits downstream expression of the primary ORF. In some embodiments, at least 1, 2, 3, 4, 5, 6, 7, 8, 9, 10, 11, 12, 13, 14, 15, 16, 17, 18, 19, 20 or more uORF were placed upstream of the nucleic acid molecule encoding the endoribonuclease. In some embodiments, the nucleic acid molecule encoding the uORF comprises a nucleotide sequence of ACCATGGGTTGA (SEQ ID NO: 1). In some embodiments, the placement of the uORF upstream of the endoribonuclease fine tunes the expression level of the endoribonucleases, and further fine tunes the expression level of the output molecule encoded by the second transcription unit.

In some embodiments, the second transcription unit of the iFFL comprises a second promoter operably linked to a nucleic acid molecule encoding an output molecule, and an endoribonuclease target site located within the 5′ untranslated region (UTR) of the nucleic acid molecule encoding the output molecule. An endoribonuclease target site, as used herein, refers to a ribonucleotide sequence that is recognized, bound, and cleaved by the endoribonuclease. The recognition site for an endoribonuclease may be 4-20 nucleotides long. For example, the RNA cleavage site may be 4, 5, 6, 7, 8, 9, 10, 11, 12, 13, 14, 15, 16, 17, 18, 19, or 20 nucleotides long. In some embodiments, endoribonuclease cleavage sites that are shorter than 4 ribonucleotides or longer than 20 nucleotides are used. In some embodiments, the endoribonuclease target site in the second transcription unit of the iFFL is capable of being cleaved by the endoribonuclease encoded by the first transcription unit of the iFFL. In some embodiments, cleavage of the endoribonuclease target site at the 5′ untranslated region (UTR) of the nucleic acid molecule encoding the output molecule represses the expression of the output molecule. In some embodiments, cleavage of the endoribonuclease target site at the 5′ untranslated region (UTR) of the nucleic acid molecule encoding the output molecule lowers the expression of the output molecule by at least 1%, 2%, 5%, 10%, 20%, 30%, 40%, 50%, 60%, 70%, 80%, 90%, or 100%.

An “output molecule,” as used herein, refers to a downstream molecule produced by the second transcription unit of the iFFL. In some embodiments, the output molecule has a basal expression level and the expression level decreases (e.g., by at least 20% relative to the basal expression level) when the endoribonuclease target cite is cleaved by the endoribonuclease expressed by the first transcription unit of the iFFL. In some embodiments, the expression level of the output molecule may be at least 20%, at least 30%, at least 40%, at least 50%, at least 60%, at least 70%, at least 80%, at least 90%, or at least 100% lower relative to the basal expression level.

The output molecule, in some embodiments, is a detectable protein. In some embodiments, a detectable protein is a fluorescent protein. A fluorescent protein is a protein that emits a fluorescent light when exposed to a light source at an appropriate wavelength (e.g., light in the blue or ultraviolet range). Suitable fluorescent proteins that may be used in accordance with the present disclosure include, without limitation, eYFP, mKO2, TagBFP, eGFP, eCFP, mKate2, mCherry, mPlum, mGrape2, mRaspberry, mGrape 1, mStrawberry, mTangerine, mBanana, and mHoneydew. In some embodiments, a detectable protein is an enzyme that hydrolyzes a substrate to produce a detectable signal (e.g., a chemiluminescent signal). Such enzymes include, without limitation, beta-galactosidase (encoded by LacZ), horseradish peroxidase, or luciferase. In some embodiments, the output molecule is a fluorescent RNA. A fluorescent RNA is an RNA aptamer that emits a fluorescent light when bound to a fluorophore and exposed to a light source at an appropriate wavelength (e.g., light in the blue or ultraviolet range). Suitable fluorescent RNAs that may be used as an output molecule in the sensor circuit of the present disclosure include, without limitation, Spinach and Broccoli (e.g., as described in Paige et al., Science Vol. 333, Issue 6042, pp. 642-646, 2011).

In some embodiments, the output molecule is a therapeutic molecule. A “therapeutic molecule” is a molecule that has therapeutic effects on a disease or condition, and may be used to treat a diseases or condition. Therapeutic molecules of the present disclosure may be nucleic acid-based or protein or polypeptide-based.

In some embodiments, nucleic acid-based therapeutic molecule may be an RNA interference (RNAi) molecule (e.g., a microRNA, siRNA, or shRNA) or an nucleic acid enzyme (e.g., a ribozyme). RNAi molecules and their use in silencing gene expression are familiar to those skilled in the art. In some embodiments, the RNAi molecule targets an oncogene. An oncogene is a gene that in certain circumstances can transform a cell into a tumor cell. An oncogene may be a gene encoding a growth factor or mitogen (e.g., c-Sis), a receptor tyrosine kinase (e.g., EGFR, PDGFR, VEGFR, or HER2/neu), a cytoplasmic tyrosine kinase (e.g., Src family kinases, Syk-ZAP-70 family kinases, or BTK family kinases), a cytoplasmic serine/threonine kinase or their regulatory subunits (e.g., Raf kinase or cyclin-dependent kinase), a regulatory GTPase (e.g., Ras), or a transcription factor (e.g., Myc). One skilled in the art is familiar with genes that may be targeted for the treatment of cancer.

Non-limiting examples of protein or polypeptide-based therapeutic molecules include enzymes, regulatory proteins (e.g., immuno-regulatory proteins), antigens, antibodies or antibody fragments, and structural proteins. In some embodiments, the protein or polypeptide-based therapeutic molecules are for cancer therapy.

Suitable enzymes (for operably linking to a synthetic promoter) for some embodiments of this disclosure include, for example, oxidoreductases, transferases, polymerases, hydrolases, lyases, synthases, isomerases, and ligases, digestive enzymes (e.g., proteases, lipases, carbohydrases, and nucleases). In some embodiments, the enzyme is selected from the group consisting of lactase, beta-galactosidase, a pancreatic enzyme, an oil-degrading enzyme, mucinase, cellulase, isomaltase, alginase, digestive lipases (e.g., lingual lipase, pancreatic lipase, phospholipase), amylases, cellulases, lysozyme, proteases (e.g., pepsin, trypsin, chymotrypsin, carboxypeptidase, elastase,), esterases (e.g. sterol esterase), disaccharidases (e.g., sucrase, lactase, beta-galactosidase, maltase, isomaltase), DNases, and RNases.

Non-limiting examples of antibodies and fragments thereof include: bevacizumab (AVASTIN®), trastuzumab (HERCEPTIN®), alemtuzumab (CAMPATH®, indicated for B cell chronic lymphocytic leukemia,), gemtuzumab (MYLOTARG®, hP67.6, anti-CD33, indicated for leukemia such as acute myeloid leukemia), rituximab (RITUXAN®), tositumomab (BEXXAR®, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX®, indicated for ovarian cancer), edrecolomab (PANOREX®), daclizumab (ZENAPAX®), palivizumab (SYNAGIS®, indicated for respiratory conditions such as RSV infection), ibritumomab tiuxetan (ZEVALIN®, indicated for Non-Hodgkin's lymphoma), cetuximab (ERBITUX®), MDX-447, MDX-22, MDX-220 (anti-TAG-72), IOR-05, IOR-T6 (anti-CD1), IOR EGF/R3, celogovab (ONCOSCINT® OV103), epratuzumab (LYMPHOCIDE®), pemtumomab (THERAGYN®), Gliomab-H (indicated for brain cancer, melanoma). In some embodiments, the antibody is an antibody that inhibits an immune check point protein, e.g., an anti-PD-1 antibody such as pembrolizumab (KEYTRUDA®) or nivolumab (OPDIVO®), or an anti-CTLA-4 antibody such as ipilimumab (YERVOY®). Other antibodies and antibody fragments may be operably linked to a promoter, as provided herein.

A regulatory protein may be, in some embodiments, a transcription factor or a immunoregulatory protein. Non-limiting, exemplary transcriptional factors include: those of the NFkB family, such as Rel-A, c-Rel, Rel-B, p50 and p52; those of the AP-1 family, such as Fos, FosB, Fra-1, Fra-2, Jun, JunB and JunD; ATF; CREB; STAT-1, -2, -3, -4, -5 and -6; NFAT-1, -2 and -4; MAF; Thyroid Factor; IRF; Oct-1 and -2; NF-Y; Egr-1; and USF-43, EGR1, Sp1, and E2F1. Other transcription factors may be operably linked to a promoter, as provided herein.

As used herein, an immunoregulatory protein is a protein that regulates an immune response. Non-limiting examples of immunoregulatory include: antigens, adjuvants (e.g., flagellin, muramyl dipeptide), cytokines including interleukins (e.g., IL-2, IL-7, IL-15 or superagonist/mutant forms of these cytokines), IL-12, IFN-gamma, IFN-alpha, GM-CSF, FLT3-ligand), and immunostimulatory antibodies (e.g., anti-CTLA-4, anti-CD28, anti-CD3, or single chain/antibody fragments of these molecules). Other immunoregulatory proteins may be operably linked to a promoter, as provided herein.

As used herein, an antigen is a molecule or part of a molecule that is bound by the antigen-binding site of an antibody. In some embodiments, an antigen is a molecule or moiety that, when administered to or expression in the cells of a subject, activates or increases the production of antibodies that specifically bind the antigen. Antigens of pathogens are well known to those of skill in the art and include, but are not limited to parts (coats, capsules, cell walls, flagella, fimbriae, and toxins) of bacteria, viruses, and other microorganisms. Examples of antigens that may be used in accordance with the disclosure include, without limitation, cancer antigens, self-antigens, microbial antigens, allergens and environmental antigens. Other antigens may be operably linked to a promoter, as provided herein.

In some embodiments, the antigen of the present disclosure is a cancer antigen. A cancer antigen is an antigen that is expressed preferentially by cancer cells (i.e., it is expressed at higher levels in cancer cells than on non-cancer cells) and, in some instances, it is expressed solely by cancer cells. Cancer antigens may be expressed within a cancer cell or on the surface of the cancer cell. Cancer antigens that may be used in accordance with the disclosure include, without limitation, MART-1/Melan-A, gp100, adenosine deaminase-binding protein (ADAbp), FAP, cyclophilin b, colorectal associated antigen (CRC)—0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain and CD20. The cancer antigen may be selected from the group consisting of MAGE-Al, MAGE-A2, MAGE-A3, MAGE-A4, MAGE-A5, MAGE-A6, MAGE-A7, MAGE-A8, MAGE-A9, MAGE-A10, MAGE-All, MAGE-Al2, MAGE-Xp2 (MAGE-B2), MAGE-Xp3 (MAGE-B3), MAGE-Xp4 (MAGE-B4), MAGE-C1, MAGE-C2, MAGE-C3, MAGE-C4 and MAGE-05. The cancer antigen may be selected from the group consisting of GAGE-1, GAGE-2, GAGE-3, GAGE-4, GAGE-5, GAGE-6, GAGE-7, GAGE-8 and GAGE-9. The cancer antigen may be selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pme1117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, PIA, EBV-encoded nuclear antigen (EBNA)-1, brain glycogen phosphorylase, SSX-1, SSX-2 (HOM-MEL-40), SSX-3, SSX-4, SSX-5, SCP-1 and CT-7, CD20 and c-erbB-2. Other cancer antigens may be operably linked to a promoter, as provided herein.

In some embodiments, a protein or polypeptide-based therapeutic molecule is a fusion protein. A fusion protein is a protein comprising two heterologous proteins, protein domains, or protein fragments, that are covalently bound to each other, either directly or indirectly (e.g., via a linker), via a peptide bond. In some embodiments, a fusion protein is encoded by a nucleic acid comprising the coding region of a protein in frame with a coding region of an additional protein, without intervening stop codon, thus resulting in the translation of a single protein in which the proteins are fused together.

In some embodiments, the output molecule is a functional molecule. A “functional molecule” refers to a molecule that is able to interact with other molecules or circuits to exert a function (e.g., transcription regulation, DNA or RNA cleavage, or any enzymatic activities). Exemplary functional molecules include, without limitation, enzymes (e.g., without limitation, nucleases), transcriptional regulators (e.g., without limitation, activators and repressors), RNAi molecules (e.g., without limitation, siRNA, miRNA, shRNA), and antibodies. In some embodiments, the functional molecule is a nuclease (e.g., a site-specific nuclease such as Csy4, Cas6, CasE, and Cse3). In some embodiments, the functional molecule is a transcriptional repressor (e.g., without limitation, TetR, CNOT7, DDX6, PPR10, and L7Ae). In some embodiments, having a functional molecule as the output molecule of the cleavage-induced transcript stabilizers described herein allows the cleavage-induced transcript stabilizer to further interact with downstream genetic circuits that contain elements responsive to the functional molecule produced by the cleavage-induced transcript stabilizer. Thus, “layering” of genetic circuits can be achieved, allowing multiple levels of complex regulation.

In some embodiments, the first promoter and the second promoter are tidentical. By having identical promoters, the transcription of the first transcription unit and the second transcription unit are coupled via transcriptional resources, thereby rendering both transcription units of the iFFL being equally affected by transcription disturbance in the environment. In some embodiments, the first promoter and the second promoter share the same transcriptional resources, such as transcription coactivator proteins (CoAs) and/or general TFs (GTFs). In some embodiments, the first promoter and the second promoter are not identical. In some embodiments, the first promoter is at least 70%, at least 80%, at least 85%, at least 90%, at least 95%, at least 96%, at least 97%, at least 98%, at least 99% or more identical to the second promoter. In some embodiments, when the first promoter and the second promoter are not identical, the iFFL is capable of adaptation to the available copy numbers of the first transcription unit and the second transcription unit. In some embodiments, the first promoter and the second promoter are constitutive promoters. In some embodiments, the first promoter and the second promoter are inducible promoters, such as promoters inducible by small molecules. For example, one of the promoters could be a small molecule-inducible promoter, which can be used to tune robustness/output levels in a manner similar to the tuning of robustness/output levels with uORFs as described elsewhere herein. In some embodiments, the first promoter and the second promoter are tissue specific promoters, such as tissue responsive promoters that are differentially responsive in different tissues. Using tissue responsive promoters provides that the expression level that is set by the iFFL varies across cell types in a directed manner. Furthermore, an inducible promoter driving a CRISPR-associated endoribonuclease such as CasE enables turning the controller off and on: when off, the output is expressed constitutively and at a high level; when on, the output is expressed at the selected controlled level.

A “promoter” refers to a control region of a nucleic acid sequence at which initiation and rate of transcription of the remainder of a nucleic acid sequence are controlled. A promoter drives expression or drives transcription of the nucleic acid sequence that it regulates. A promoter may also contain sub-regions at which regulatory proteins and molecules may bind, such as RNA polymerase and other transcription factors. Promoters may be constitutive, inducible, activatable, repressible, tissue-specific or any combination thereof. A promoter is considered to be “operably linked” when it is in a correct functional location and orientation in relation to a nucleic acid sequence it regulates to control (“drive”) transcriptional initiation and/or expression of that sequence. In some embodiments, the first promoter and the second promoter in the iFFL are inducible promoters or constitutive promoter.

In some embodiments, a promoter is a constitutive promoter. Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al., Cell, 41:521-530 (1985)), the SV40 promoter, the dihydrofolate reductase promoter, the β-actin promoter, the phosphoglycerol kinase (PGK) promoter, and the EF1α promoter [Invitrogen]. In some embodiments, a promoter is an enhanced chicken β-actin promoter.

In some embodiments, a promoter is an “inducible promoter,” which refer to a promoter that is characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter. An inducible promoter of the present disclosure may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof. Inducible promoters of the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some embodiments, the first and the second transcription unit of the iFFL are present on the same nucleic acid. In some embodiments, the first and the second transcription unit of the iFFL are present on different nucleic acids. In some embodiments, the first and the second transcription unit of the iFFL are present on the same vector. In some embodiments, the first and the second transcription unit of the iFFL are present on different vectors.

Also provided herein are nucleic acid(s) and vector(s) comprising the engineered iFFL described herein. Each component of the engineered iFFL may be included in one or more (e.g., 2, 3 or more) nucleic acid molecules (e.g., vectors) and introduced into a cell. A “nucleic acid” is at least two nucleotides covalently linked together, and in some instances, may contain phosphodiester bonds (e.g., a phosphodiester “backbone”). A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine. Nucleic acids of the present disclosure may be produced using standard molecular biology methods (see, e.g., Green and Sambrook, Molecular Cloning, A Laboratory Manual, 2012, Cold Spring Harbor Press).

In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

In some embodiments, the engineered iFFL is delivered to a cell by one or more vectors. A “vector” refers to a nucleic acid (e.g., DNA) used as a vehicle to artificially carry genetic material (e.g., an engineered nucleic acid) into a cell where, for example, it can be replicated and/or expressed. In some embodiments, a vector is an episomal vector (see, e.g., Van Craenenbroeck K. et al. Eur. J. Biochem. 267, 5665, 2000). A non-limiting example of a vector is a plasmid, RNA replicons, viral vectors (e.g., rAAV, lentivirus). Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector (e.g., retrovirus, adenovirus, adeno-associated virus, helper-dependent adenovirus systems, hybrid adenovirus systems, herpes simplex virus, pox virus, lentivirus, Epstein-Barr virus). In some embodiments, the viral vector is derived from an adeno-associated virus (AAV). In some embodiments, the viral vector is derived from an herpes simplex virus (HSV).

The nucleic acids or vectors containing the transcription units of the engineered iFFL may be delivered to a cell by any methods known in the art for delivering nucleic acids. For example, for delivering nucleic acids to a prokaryotic cell, the methods include, without limitation, transformation, transduction, conjugation, and electroporation. For delivering nucleic acids to a eukaryotic cell, methods include, without limitation, transfection, electroporation, and using viral vectors. In some embodiments, the first transcription unit of the iFFL and the second transcription unit of the iFFL are delivered to the cell by different nucleic acids or vectors. In some embodiments, there are different copy numbers of the first transcription unit and the second transcription unit. In some embodiments, the ratio between the first transcription unit and the second transcription unit is proportional. Proportional delivery of the first and the second transcription unit of the iFFL means they are delivered at a ratio. In some embodiments, the ration between the nucleic acids or vectors carrying the first transcription unit of the iFFL and the nucleic acids or vectors carrying the second transcription unit is 1:1, 1:2, 1:3, 1:4, 1:5, 1:6, 1:7, 1:8, 1:9, 1:10, 2:3, 2:5, 2:7, 2:9, 3:4, 3:5, 3:7, 3:8, 3:10, 4:5, 4:7, 4:9, 4:10, 5:6, 5:7, 5:8, 5:9, 6:7, 7:8, 7:9, 7:10, 8:9, 9:10, 10:1, 9:1, 8:1, 7:1, 6:1, 5:1, 4:1, 3:1, 2:1, 3:2, 5:2, 7:2, 9:2, 4:3, 5:3, 7:3, 8:3, 10:3, 5:4, 7:4, 9:4, 10:4, 6:5, 7:5, 8:5, 9:5, 7:6, 8:7, 9:7, 10:7, 9:8, or 10:9.

Also provided herein are the cells comprising the engineered iFFL or the vectors encoding the same as described herein. A “cell” is the basic structural and functional unit of all known independently living organisms. It is the smallest unit of life that is classified as a living thing. Some organisms, such as most bacteria, are unicellular (consist of a single cell). Other organisms, such as humans, are multicellular.

In some embodiments, a cell for use in accordance with the present disclosure is a prokaryotic cell, which may comprise a cell envelope and a cytoplasmic region that contains the cell genome (DNA) and ribosomes and various sorts of inclusions. In some embodiments, the cell is a bacterial cell. As used herein, the term “bacteria” encompasses all variants of bacteria, for example, prokaryotic organisms and cyanobacteria. Bacteria are small (typical linear dimensions of around 1 micron), non-compartmentalized, with circular DNA and ribosomes of 70S. The term bacteria also includes bacterial subdivisions of

Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are gram-negative cells, and in some embodiments, the bacterial cells are gram-positive cells. Examples of bacterial cells that may be used in accordance with the invention include, without limitation, cells from Yersinia spp., Escherichia spp., Klebsiella spp., Bordetella spp., Neisseria spp., Aeromonas spp., Francisella spp., Corynebacterium spp., Citrobacter spp., Chlamydia spp., Haemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., and/or Stremtomyces spp. In some embodiments, the bacterial cells are from Staphylococcus aureus, Bacillus subtilis, Clostridium butyricum, Brevibacterium lactofermentum, Streptococcus agalactiae, Lactococcus lactis, Leuconostoc lactis, Streptomyces, Actinobacillus actinobycetemcomitans, Bacteroides, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphlococcus lugdunensis, Leuconostoc oenos, Corynebacterium xerosis, Lactobacillus plantarum, Streptococcus faecalis, Bacillus coagulans, Bacillus cereus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechrornogenes, Streptomyces ghanaenis, Halobacterium strain GRB, or Halobaferax sp. strain Aa2.2.

In some embodiments, a cell for use in accordance with the present disclosure is a eukaryotic cell, which comprises membrane-bound compartments in which specific metabolic activities take place, such as a nucleus. Examples of eukaryotic cells for use in accordance with the invention include, without limitation, mammalian cells, insect cells, yeast cells (e.g., Saccharomyces cerevisiae) and plant cells. In some embodiments, the eukaryotic cells are from a vertebrate animal. In some embodiments, the cell is a mammalian cell. In some embodiments, the cell is a human cell. In some embodiments, the cell is from a rodent, such as a mouse or a rat. Examples of vertebrate cells for use in accordance with the present disclosure include, without limitation, reproductive cells including sperm, ova and embryonic cells, and non-reproductive cells, immune, kidney, lung, spleen, lymphoid, cardiac, gastric, intestinal, pancreatic, muscle, bone, neural, brain and epithelial cells. Stem cells, including embryonic stem cells or induced pluripotent stem cells, can also be used.

In some embodiments, the cell is a diseased cell. A “diseased cell,” as used herein, refers to a cell whose biological functionality is abnormal, compared to a non-diseased (normal) cell. In some embodiments, the diseased cell is a cancer cell.

In some embodiments, the cell is a cell used for recombinant protein production. Non-limiting examples of recombinant protein producing cells are Chinese hamster ovary (CHO) cells, human embryonic kidney (HEK)-293 cells, verda reno (VERO) cells, nonsecreting null (NS0) cells, human embryonic retinal (PER.C6) cells, Sp2/0 cells, baby hamster kidney (BHK) cells, Madin-Darby Canine Kidney (MDCK) cells, Madin-Darby Bovine Kidney (MDBK) cells, and monkey kidney CV1 line transformed by SV40 (COS) cells.

In some embodiments, the engineered iFFL is inserted into the genome of the cell. Methods of inserting genetic circuits into the genome of a cell are known to those skilled in the art (e.g., via site-specific recombination, using any of the known genome-editing tools, or using other recombinant DNA technology). In some instances, integrating the cleavage-induced transcript stabilizer into the genome of a cell is advantageous for its applications (e.g., therapeutic application or biomanufacturing application), compared to a cell engineered to simply express a transgene (e.g., via transcription regulation). It is known that genetically engineered cells suffer from epigenetic silencing of the integrated transgene. However, continuous transcription of transgenes helps to prevent their silencing, which is not possible with transcriptionally-regulated gene circuits relying on transcriptional repression. In contrast, the cleavage-induced transcript stabilizer described herein relies on RNA-level regulation and can achieve continuous transcription of the transgenes.

Also provided herein are animals comprising the engineered iFFL, the vectors encoding the same, or the cells comprising the engineered iFFL as described herein. In some embodiments, the non-human animal is a mammal. Non-limiting examples of non-human mammals are: mouse, rat, goat, cow, sheep, donkey, cat, dog, camel, or pig.

### II. Pharmaceutical Composition

In some aspects, the present disclosure, at least in part, relates to a pharmaceutical composition, comprising the engineered iFFL, the vector comprising the same, the cells, as described herein. The pharmaceutical composition described herein may further comprise a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used for in vivo administration must be sterile. This is readily accomplished by, for example, filtration through sterile filtration membranes. The pharmaceutical compositions described herein may be placed into a container having a sterile access port, for example, an intravenous solution bag or vial having a stopper pierceable by a hypodermic injection needle.

In other embodiments, the pharmaceutical compositions described herein can be formulated for intra-muscular injection, intravenous injection, intratumoral injection or subcutaneous injection.

The pharmaceutical compositions described herein to be used in the present methods can comprise pharmaceutically acceptable carriers, buffer agents, excipients, salts, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some examples, the pharmaceutical composition described herein comprises lipid nanoparticles which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In other examples, the pharmaceutical composition described herein can be formulated in sustained-release format. Suitable examples of sustained-release preparations include semipermeable matrices of solid hydrophobic polymers containing the engineered iFFL, the vector comprising the same, or the cell comprising the same, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(-)-3-hydroxybutyric acid.

Suitable surface-active agents include, in particular, non-ionic agents, such as polyoxyethylenesorbitans (e.g., TWEEN™ 20, 40, 60, 80 or 85) and other sorbitans (e.g., SPAN™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Suitable emulsions may be prepared using commercially available fat emulsions, such as INTRALIPID™, LIPOSYN™, INFONUTROL™, LIPOFUNDIN™ and LIPIPHYSAN™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets having a suitable size and can have a pH in the range of 5.5 to 8.0.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### III. Applications

The present disclosure, at least in part, relates to the use of the engineered iFFL described herein.

In some embodiments, the present disclosure provides a method of delivering an output molecule to a subject in need thereof, the method comprising administering to a subject in need thereof the engineered iFFL, the cell comprising the same, or the composition comprising the same.

In some embodiments, the present disclosure provides a method of delivering an output molecule to a cell, the method comprising contacting the cell the engineered iFFL, the cell comprising the same, or the composition comprising the same.

In some embodiments, the present disclosure provides a method of maintaining expression level of an output molecule to transcription disturbance, the method comprising administering to the subject the engineered iFFL, the cell comprising the same, or the composition comprising the same. If an output molecule is not delivered by an engineered iFFL described herein, the transcription disturbance (e.g., transcription resource, off-target promoter interaction, or DNA copy number) would affect the expression level of the output molecule drastically. If an output molecule is delivered by the iFFL described herein, the expression level of the output molecule is capable of being maintained at a certain level (e.g., less than 20%, 15%, 10%, 9%, 8%, 7%, 6%, 5%, 4%, 3%, 2% or 1% variation) in response to transcription disturbance.

In some embodiments, the first transcription unit and the second transcription unit are delivered on the same nucleic acid. In some embodiments, the first transcription unit and the second transcription unit are delivered on the same vector. In some embodiments, the first transcription unit and the second transcription unit are delivered on different nucleic acids. In some embodiments, different copy numbers of the first transcription unit and the second transcription unit are delivered. In some embodiments, the first transcription unit and the second transcription unit are delivered on the different vectors. In some embodiments, the ratio between the first transcription unit and the second transcription unit is proportional.

In some embodiments, the administration of the engineered iFFL is performed once in a lifetime, once every 10 years, once every 5 years, once every year, once every six month or once a month.

The engineered iFFL, the vector, the cells and the pharmaceutical composition described herein, can be used to treat various diseases (e.g., diseases treatable by the therapeutic molecules produced by the engineered iFFL).

To practice the method disclosed herein, an effective amount of any of the pharmaceutical compositions described herein can be administered to a subject (e.g., a human) in need of the treatment via a suitable route, such as intratumoral administration, by intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, pharmaceutical composition described herein can be aerosolized using a fluorocarbon formulation and a metered dose inhaler, or inhaled as a lyophilized and milled powder. In some examples, the pharmaceutical composition described herein is formulated for intratumoral injection. In particular examples, the pharmaceutical composition may be administered to a subject (e.g., a human patient) via a local route, for example, injected to a local site such as a tumor site or an infectious site.

As used herein, “an effective amount” refers to the amount of each active agent required to confer therapeutic effect on the subject, either alone or in combination with one or more other active agents. For example, the therapeutic effect can be reduced tumor burden, reduction of cancer cells, increased immune activity, reduction of a mutated protein, reduction of over-active immune response. Determination of whether an amount of engineered iFFL achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations of pharmaceutical composition described herein may be appropriate. Various formulations and devices for achieving sustained release are known in the art.

In some embodiments, the treatment is a single injection of the pharmaceutical composition described herein. In some embodiments, the method described herein comprises administering to a subject in need of the treatment (e.g., a human patient) one or multiple doses of pharmaceutical composition described herein.

In some example, dosages for a pharmaceutical composition described herein may be determined empirically in individuals who have been given one or more administration(s) of the pharmaceutical composition. Individuals are given incremental dosages of the synthetic pharmaceutical composition described herein. To assess efficacy of the engineered iFFL, an indicator of the disease/disorder can be followed. For repeated administrations over several days or longer, depending on the condition, the treatment is sustained until a desired suppression of symptoms occurs or until sufficient therapeutic levels are achieved to alleviate a target disease or disorder, or a symptom thereof.

In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen of the pharmaceutical composition described herein used can vary over time.

For the purpose of the present disclosure, the appropriate dosage of the pharmaceutical composition described herein will depend on the specific miRNA signature of the cell and the miRNA to be expressed, the type and severity of the disease/disorder, the pharmaceutical composition described herein is administered for preventive or therapeutic purposes, previous therapy, the patient's clinical history and response to the engineered iFFL, and the discretion of the attending physician. A clinician may administer a pharmaceutical composition described herein, until a dosage is reached that achieves the desired result. Methods of determining whether a dosage resulted in the desired result would be evident to one of skill in the art. Administration of one or more pharmaceutical composition described herein can be continuous or intermittent, depending, for example, upon the recipient's physiological condition, whether the purpose of the administration is therapeutic or prophylactic, and other factors known to skilled practitioners. The administration pharmaceutical composition described herein may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

The subject to be treated by the methods described herein can be a mammal, such as a human, farm animals, sport animals, pets, primates, horses, dogs, cats, mice and rats. In one embodiment, the subject is a human.

In some embodiments, the subject may be a human patient having, suspected of having, or at risk for a disease. Non-limiting examples of diseases that are suitable for engineered iFFL based therapy are: Alpha-1 antitrypsin deficiency, Hypercholesterolemia, Hepatitis B infection, Liver adenoma due to HIV infection, Hepatitis C virus infection, Ornithine transcarbamylase deficiency, Hepatocellular carcinoma, Amyotrophic lateral sclerosis, Spinocerebellar ataxia type 1, Huntington's disease, Parkinson disease, Spinal and Bulbar muscular atrophy, Pyruvate dehydrogenase deficiency, Hyperplasia, obesity, facioscapulohumeral muscular dystrophy (FSHD), Nerve Injury-induced Neuropathic Pain, Age-related macular degeneration, Retinitis pigmentosa, heart failure, cardiomyopathy, cold-induced cardiovascular dysfunction, Asthma, Duchenne muscular dystrophy, infectious diseases, or cancer.

Non limiting examples of cancers include melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, gastric cancer, and various types of head and neck cancer, including squamous cell head and neck cancer. In some embodiments, the cancer can be melanoma, lung cancer, colorectal cancer, renal-cell cancer, urothelial carcinoma, or Hodgkin's lymphoma.

A subject having a target disease or disorder (e.g., cancer or an infectious disease) can be identified by routine medical examination, e.g., laboratory tests, organ functional tests, CT scans, or ultrasounds. A subject suspected of having any of such target disease/disorder might show one or more symptoms of the disease/disorder. A subject at risk for the disease/disorder can be a subject having one or more of the risk factors associated with that disease/disorder. Such a subject can also be identified by routine medical practices.

In some embodiments, a pharmaceutical composition described herein may be co-used with another suitable therapeutic agent (e.g., an anti-cancer agent an anti-viral agent, or an anti-bacterial agent) and/or other agents that serve to enhance effect of an engineered iFFL. In such combined therapy, the pharmaceutical composition described herein, and the additional therapeutic agent (e.g., an anti-cancer therapeutic agent or others described herein) may be administered to a subject in need of the treatment in a sequential manner, i.e., each therapeutic agent is administered at a different time. Alternatively, these therapeutic agents, or at least two of the agents, are administered to the subject in a substantially simultaneous manner. Combination therapy can also embrace the administration of the agents described herein in further combination with other biologically active ingredients (e.g., a different anti-cancer agent) and non-drug therapies (e.g., surgery).

IV. General techniques

The practice of the present invention will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A laboratory notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Introduction

A significant goal of synthetic biology is to develop genetic devices for accurate and robust control of gene expression. Lack of modularity, wherein a device output does not depend uniquely on its intended inputs but also on its context, leads to poorly predictable device behavior. One contributor to lack of modularity is competition for shared limited gene expression resources, which can induce ‘coupling’ between otherwise independently-regulated genes. Here the effects of resource competition on engineered genetic systems in mammalian cells was quantified, and a feedforward controller to make gene expression robust to changes in resource availability was developed. In addition to mitigating resource competition, the feedforward controller described herein also enables adaptation to multiple log-orders of DNA copy number variation and is predictably tunable with upstream open reading frames. The resource competition characterization along with the feedforward control device will be critical for achieving robust and accurate control of gene expression.

A promising strategy for engineering complex genetic devices is to compose together simpler systems that have been characterized in isolation. A critical assumption of this modular design approach is that subsystems maintain their input/output (i/o) behavior when assembled into larger systems. However, this assumption often fails due to context dependence, i.e., the behavior of a module depends on the surrounding systems. There are many sources of context-dependence, including unexpected off-target interactions between regulators and promoters, transcription factor loading by DNA targets6, gene orientation, and resource loading by expressed genes. To date, much effort has gone into identifying gene regulators with unique binding specificity, e.g. between transcription factors (TFs) and their DNA binding sites. Unique binding specificity enables gene regulators to work orthogonally, since they do not directly interfere with each other's binding and regulation. Nevertheless, even if subsystems are entirely composed of orthogonal regulators, they can become coupled with each other via competition for shared cellular resources. For example, it has been demonstrated in prokaryotes that genes compete for the usage of ribosomes, such that increased expression from one gene decreases expression from others by sequestering (i.e. loading) the ribosome. Little work has been done to understand how resource competition affects engineered genetic devices in eukaryotic cells. Furthermore, while solutions to the ribosome resource competition problem in bacterial cells have appeared recently, solutions to resource competition in mammalian cells are still missing.

In mammalian cells, loading of several types of cellular resources shared among multiple genes has been shown to affect gene expression, including splicing factors, miRNA processing factors, RISC complexes, and the proteasome. A potent form of resource competition called ‘squelching’ occurs when transcriptional activators (TAs) or strong promoters sequester transcription coactivator proteins (CoAs) and/or general TFs (GTFs), reducing transcription of other genes. At sufficiently high expression levels of a given TA, these transcriptional resources are sequestered even from the TA molecules bound to the target promoter, yielding a bell-like dose-response curve, where the expression of the TA's target gene peaks at an intermediate level of TA and then decreases as the TA concentration is further increased (often referred to as ‘self-squelching’). As many established synthetic eukaryotic gene regulation systems utilize TAs, squelching represents a potentially pervasive problem in the space of eukaryotic genetic engineering. Here, competition for gene expression resources is considered as a long-lasting problem, requiring further investigation of the quantitative consequences of resource competition on mammalian genetic circuits.

General transcription factors (also referred to herein as general TFs or GTFs) bind to promoters on a DNA sequence and/or form a transcription preinitiation complex that participates in activation of transcription. In bacteria, general transcription factors include sigma factors, such as σ70 (RpoD), σ19 (FecI), σ24 (RpoE), σ28 (RpoF/FliA), σ32 (RpoH), σ38 (RpoS), and σ54 (RpoN). In eukaryotes (and archaea), general transcription factors include, for transcription initiation by eukaryotic RNA polymerase II: Gaffs, TFIIB, TFIID, TFIIE, TFIIF, and TFIIH. A transcription preinitiation complex in eukaryotes also includes TATA binding protein (TBP).

An experimental model system was developed to recapitulate the effects of transcriptional resource competition and provide in-depth characterization of these effects. This model system was then used to evaluate the performance of a feedforward controller designed to cancel the effects of resource loading on gene expression. Specifically, this model system utilizes Gal4 TAs of varying strength to measure the extent to which TA expression sequesters transcriptional resources from non-target genes. A mathematical model that explains the effects of transcriptional resource sequestration by TAs on expression of target and non-target genes of the TA was developed. The effect of different Gal4 TAs on commonly-used constitutive promoters in various mammalian cell lines was measured to identify combinations of activators and promoters in each cell line where minimal effect on the non-target promoters is observed. Overall, our results provide extensive analysis for determining the extent to which transcriptional resource competition affects gene circuit behavior in mammalian cells.

The goal is to make the output protein level of a given genetic device insensitive to changes in available gene expression resources, including CoAs and GTFs. By doing so, effective decoupling between the behaviors of resource-coupled genetic devices and the available resources can be achieved. To approach this problem, resource availability is regarded as a disturbance input to a genetic device and a controller that can be added to any device to ‘cancel out’ the effect of resource competition on the device's output. In prokaryotes, it has been shown that quasi-integral feedback control can make the output protein level of a genetic device insensitive to changes in ribosome availability. In both prokaryotes and eukaryotes, incoherent feedforward loops (iFFLs) have been used to make gene expression levels insensitive to the copy number of a gene. Engineered herein was an iFFL using CasE, an endoribonuclease (endoRNase) from a type I CRISPR system, to make a genetic device's output insensitive to changes in the availability of transcriptional resources (FIG. 1A-1E). Through experiments in mammalian cells, the data showed that iFFLs can make the output protein level of a genetic device insensitive to variations in availability of gene expression resources. Further, iFFL design performs well in combination with different activators and cell lines, demonstrating that the iFFL described herein is general and applicable to a variety of contexts. Beyond resource competition, this iFFL design also made a genetic device's output insensitive to multiple log-order changes in gene copy number, substantially improving upon previously published miRNA-based designs. In addition, the iFFL reduced the dynamic effects of plasmid uptake and dilution on protein expression during transient transfection, thereby broadening the time window over which stable expression levels can be achieved. Overall, this iFFL design can have broad utility for engineering mammalian genetic devices which behave as predicted in a context-independent manner.

### Results

### Characterization of Transcriptional Resource Competition

Cells provide a finite pool of resources for gene expression. To express any one gene, the cell must allocate resources to this gene, thereby reducing the availability of resources to other genes. Here, the effect of this reduced availability of resources on the output of a genetic device was considered. Specifically, a genetic device as a system composed of one gene that takes regulatory inputs (e.g. sequence-specific TFs) and gives the gene's expressed RNA and/or protein as output was defined. Further, a genetic module as one or more genetic devices that were linked together by regulatory interactions were defined. Experimentally, fluorescent markers were included to measure the expression level of non-fluorescent proteins in some modules. Independently-regulated devices become implicitly coupled by competition for gene expression resources, wherein expression of a gene in one device ‘loads’ the pool of resources, thereby decreasing resource availability to other devices (FIG. 1A). Because of this coupling, the i/o behavior of a genetic device or module became dependent on the presence of other devices and modules in the cell.

Previous studies have shown that competition for transcriptional resources including coactivators (CoAs) and general transcription factors (GTFs) can reduce gene expression levels. Transcriptional activators (TAs) in eukaryotes are comprised of a DNA-binding domain (DBD) and an activation domain (AD), the latter of which recruits CoAs and/or GTFs to initiate transcription. When a given TA is in excess, the binding between the TA and CoAs/GTFs in solution and at off-target DNA loci can form unproductive complexes that sequester these factors, a phenomenon referred to as squelching20. Importantly, ADs alone, without a DBD, can also cause squelching.

Competition for transcriptional resources by different genetic devices using the genetic model system was recapitulated in FIG. 1E. The Gal4 DBD was fused to several ADs of varying potency, of which five were chosen for in-depth study: VP16, VPR, and the individual components of VPR (VP64, Rta, and p65). The model system comprises two genetic modules, each with one or more genetic devices: (i) a device with a constitutive gene: CMV: Output1 and (ii) Gal4 TA expression: hEF1a: Gal4-AD and a Gal4-activated gene: UAS: Output2 (FIG. 1E). To more precisely measure the delivery of Gal4 TAs to each cell, a fluorescent reporter (Gal4 Marker) that was co-titrated with the Gal4 TAs was included prior to transfection of all the plasmids into HEK-293FT cells. The resulting dose-response curves for knockdown of CMV: Output1 and activation of UAS: Output2 are shown in FIG. 1F. Full distributions for samples with varying Gal4 TA input levels and a comparison of the DNA input amount vs Gal4 At the highest dosage tested, all five Gal4 TAs knocked down CMV: Output1 by at least 2-fold, with Gal4-VPR causing nearly 8-fold knockdown (FIG. 1F). Each curve was similar in shape, with the main difference being the amount of Gal4 TA needed to reduce CMV-driven expression by half, which varied by over 20-fold between Gal4-VP64 and Gal4-VPR.

Resource sequestration due to addition of Gal4 TAs can occur at different stages of gene expression: (a) the expression of Gal4 itself requires both transcriptional and translational resources, (b) the action of Gal4 activating its target causes additional sequestration of both types of resources due to expression of the target gene, and (c) Gal4 directly binds to and sequesters transcriptional resources in solution and/or at off-target DNA loci24. The ability of Gal4 TAs repressing CMV transcription was validated by RT-qPCR measurement of CMV-driven mRNA levels. Indeed, CMV-driven mRNA levels were knocked down ˜2-fold by Gal4-VP16 and ˜16-fold by Gal4-VPR. In the same samples, a fraction of the cells were collected for flow cytometry to measure protein expression levels. The magnitude of knockdown of protein levels closely matched that of the mRNA levels, suggesting that most of the knockdown was caused at the transcriptional level. Additional experiments showed that VPR alone and Gal4-VPR both knock down CMV expression, but neither the Gal4 DBD nor the luminescent protein Fluc2 do so. Because these proteins were expressed by identical promoters and thus place similar demands on gene expression resources, it was concluded that (a) is negligible compared to (b) and (c). Furthermore, the knockdown of CMV expression by Ga14-VPR was similar regardless of whether the Gal4 target gene was present, indicating that in this system, (b) is small compared to (c). Thus, the AD, whether fused or not to the TA (Gal4), sequestered transcriptional resources from the CMV promoter, rendering it a major player in the observed knock down of the CMV output expression.

The dose-response curves of the Gal4 TAs activating the target gene (UAS: Output2) was also characterized. The activation dose-response curve of some activators (Gal4-Rta, Gal4-p65, and Gal4-VPR) clearly showed decreasing output at high dosages of the activators. A mathematical model was developed to provide a tool for predicting the effects of resource loading on gene circuit behavior. This model can recapitulate both non-target gene knockdown (FIG. 1F) and on-target self-squelching behavior by a TA. Interestingly, the relative UAS: Output2 between each activator was strongly dose-dependent; for example, Gal4-p65 drove ˜6-fold higher expression than Gal4-VP64 at the lowest DNA dosage, whereas Gal4-VP64 drove nearly 2-fold higher expression than Gal4-p65 at the highest DNA dosage. For further discussion of this phenomenon, model fitting, and model validation. From prior work, it is unclear whether the minimum concentration of TA necessary for maximal activation of on-target genes is sufficient to knock down non-target genes. The expression of CMV: Output1 to UAS: Output2 at each level of each Gal4 TA was compared to measure the trade-off in expression of both genes. The results showed that for each Gal4 TA, maximum UAS: Output2 expression occurred at a concentration of Gal4 that knocked down CMV: Output1 by at least 2-fold. Overall, these results indicate that for TAs to drive high levels of expression, significant knockdown of non-target genes is likely to be observed.

Finally, the results measured in transient transfection were validated to be consistent with the behavior of genetic devices integrated into the genome. To do so, HEK-293FT cells were transfected with one of two lentiviral constructs: (i) an rtTA activator and an rtTA-driven fluorescent reporter or (ii) a Gal4-driven fluorescent reporter. Following lentiviral integration, both cell lines were also transfected with Gal4 activators and found that both rtTA- and Gal4-driven expression were negatively affected by Gal4 activators at high activator dosages. The responses of the non-target tet-on system and on-target promoter were both well-predicted by the model fits from our transfection experiments. Thus, our resource competition results are extensible to genes located in various contexts.

Activator and Non-Target Promoter Combinations with Minimal Coupling

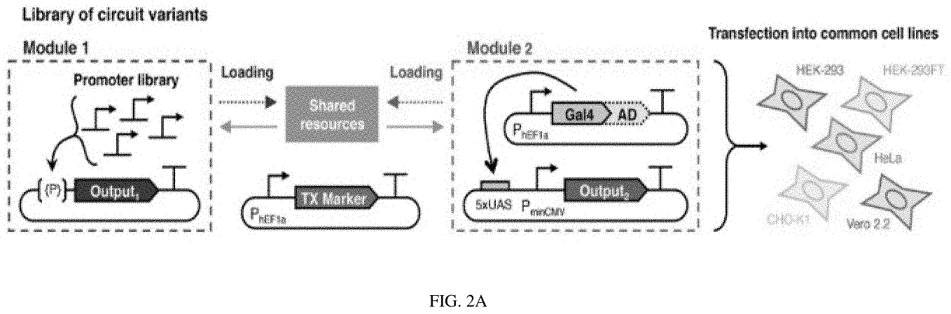

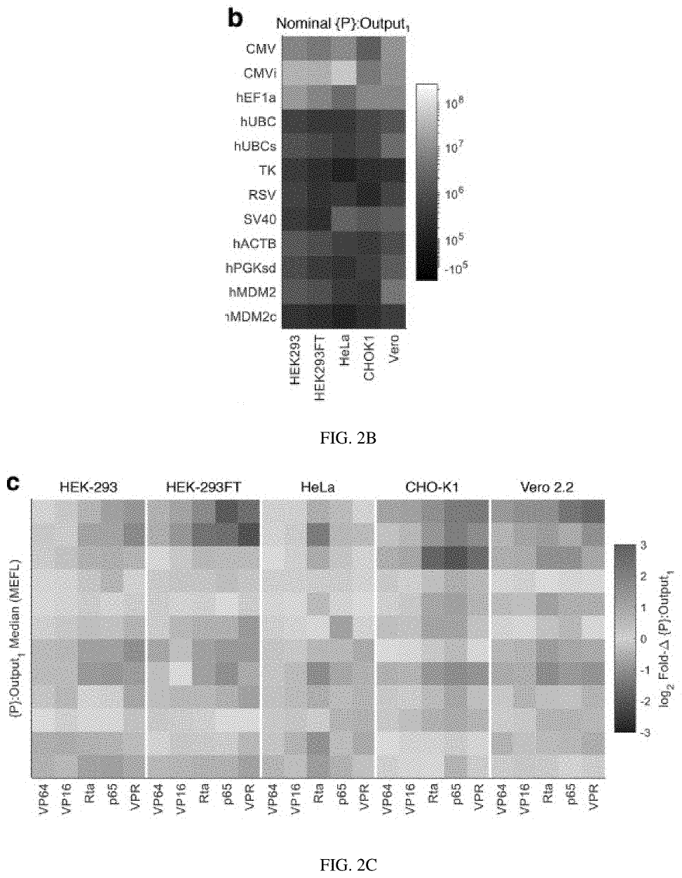

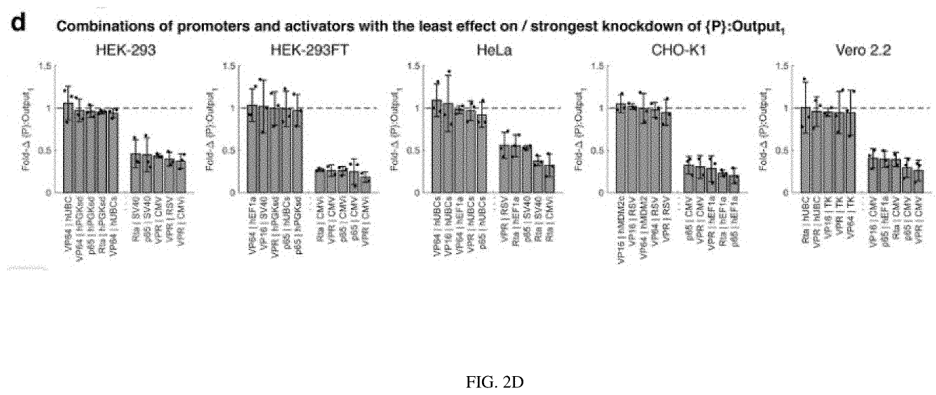

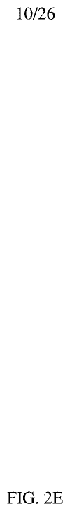

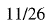

The genetic model system of FIG. 1E was extended to a library of such systems with varying non-target promoters ({P}: Output1) in Module 1 and varying ADs fused to Gal4 in Module 2, then transfected each combination of variants into different cell lines (FIG. 2A). FIG. 2B showed the nominal expression level of {P}: Output1 in Module 1 for each combination of promoter and cell line tested. The nominal expression was measured in samples co-transfected with Gal4-None, which lacks an AD and thus does not appreciably load transcriptional resources. Across this set of cell lines, the hEF1a promoter showed the most consistent nominal expression and the CMV-intron (CMVi) promoter generally showed the highest nominal expression level. The relative nominal expression level of each promoter was generally well-correlated between cell lines, with Vero cells showing higher expression overall. FIG. 2C showed the fold-changes in {P}: Output1 expression in each cell line when co-transfected with different Gal4 TAs, with the AD fused to Gal4 in each set of samples shown along the bottom of the heatmap. Decreased expression of Output1 was observed in the majority of combinations, with the strongest knockdown of Output1 observed for the CMV-based, RSV, and SV40 promoters, as well as in CHO-K1 and Vero 2.2 cells broadly. Some promoters had slightly increased expression in the presence of the Gal4 TAs. From the Output1 fold-changes in FIG. 2C, patterns that help guide design choices can be extracted for specific combinations of promoters and TAs that minimized coupling between modules due to resource competition. Comparing the Gal4 TAs, across cell lines, Gal4-VP16 and -VP64 had relatively weak effects and Gal4-Rta, -p65, and -VPR had relatively strong effects. Gal4-VP64 and -VP16 caused less than a 20% change in expression of {P}: Output1 on average across all promoters and cell lines, compared to a 30-40% reduction on average by Gal4-Rta, -p65, and -VPR. At the dosage of activators tested, Gal4-VP64 and -VP16 also tended to give the highest level of UAS: Output2.

Overall, the effects of Gal4 TAs on a given constitutive promoter were mildly correlated between cell lines, with CHO-K1 and Vero 2.2 cells experiencing repressive effects across nearly all promoters. However, there were cases where different promoters were affected more or less strongly in different cell types. For example, in both HEK cell lines, the hEF1a promoter was less affected by Gal4 TA competition than the CMV promoter, whereas in CHO-K1 cells the opposite was seen (FIG. 2C). The Gal4 TA with the strongest negative effect on a given promoter was also not necessarily the same between cell lines. For instance, Gal4-VPR typically showed the strongest knockdown in HEK cells, whereas Gal4-Rta and Gal4-p65 did so in HeLa and CHO cells, respectively. Thus, while many patterns were preserved between cell lines, the effects of resource competition in one cell line do not necessarily predict the effects in others.

The constitutive promoters tested varied more than 2 orders of magnitude in strength and originated from both viral and human DNA. Some promoters drove expression that was nearly undetectable, which was partially adjusted for by subtracting the median of the untransfected cells from that of the transfected cells prior to computing fold-changes. In general, viral promoters in Module 1 were more strongly affected by resource competition than human promoters, suggesting that they have lower affinity for GTFs/CoAs and/or that they utilize a relatively high fraction of specific CoAs that are sequestered by the Gal4 TAs. Heirarchical clustering of the fold-changes in each promoter's expression identified that in our set, human promoters are more similarly affected by Gal4 TAs than viral promoters, possibly owing to their similar CpG-island based architectures. While widespread reductions was observed and in some cases increases in Output1 in response to the Gal4 TAs, there were some combinations of promoters and Gal4 TAs in each cell line that had little to no effect. The five promoter-TA combinations with either the least effect on or strongest knockdown of Output1 are reported in FIG. 2C. In HEK-293 and HEK-293FT cells, hPGK and the hUBC promoter variants were weakly affected in combination with most Gal4 TAs, together making up 8/10 of the most unchanged combinations across both cell lines. In HeLa cells, hEF1a and the hUBC promoter variants were generally the least-affected by competition; note that the TK promoter in HeLa was omitted from this analysis because its nominal expression level was undetectable. In CHO-K1 and Vero 2.2 cells, RSV/hMDM2 variants and TK/hUBC were least affected by resource competition, respectively. Consistent with the general effects on promoters described above, the combinations of promoters and TAs with the strongest negative effects were nearly all with viral promoters. However, there were some notable exceptions; in particular, the hEF1a promoter was the most strongly knocked down promoter in CHO-K1 cells.

So far the study was focused on how coupling between the constitutive promoters (Module 1) and the Gal4 TAs (Module 2) affects expression of the promoters (Output1); however, this coupling can also work in reverse such that Gal4-driven expression (Output2) is affected by Module 1. The effects of resource sequestration by the constitutive reporters on Gal4-driven activation of UAS: Output2 was then examined. It was found that the expression UAS: Output2 was largely the same between samples with the same Gal4 TA but different promoters, but with notable exceptions in each cell line. For example, samples with the CMV or hEF1a promoters generally had higher or lower UAS: Output2 expression, respectively. In addition, samples with the hMDM2c promoter showed reduced UAS: Output2 expression in CHO-K1 cells, and samples with hUBC promoter variants showed increased UAS: Output2 expression in Vero 2.2 cells.

While it appeared that that effects from resource competition are entirely derived from the AD of a TA such as Gal4, it has been shown that reducing the strength of or eliminating DNA-DBD binding may relieve the effects of transcriptional resource loading by TAs27. Tge results showed that VPR alone, Gal4-VPR, and VPR fused to a zinc finger protein (ZFP), dCas9, and rTetR had on expression of each of the constitutive promoters tested above. It was found that the effects on each promoter followed a similar trend, with rTetR-VPR showing the most similar effects to Gal4-VPR, dCas9-VPR showing the least strong effects, and ZFP-VPR being the only variant to not knock down the CMV-based promoters. The effects on each constitutive promoter were similar for dCas9-VPR +/−gRNA as well as rTetR-VPR +/− Dox, indicating that expression by these specific promoters did not significantly load gene expression resources and that Dox or gRNA binding are not required for rTetR-VPR or dCas9-VPR to sequester resources, respectively.

Overall, the characterization results in FIGS. 2C-2D are capable of guiding choices of combinations of promoters and Gal4 TAs that minimize resource competition in engineered genetic circuits in the given mammalian cell lines.

Model-Guided design of a Resource-Decoupled Genetic Module Using an EndoRNase-Based iFFL

Though some combinations of promoters and TAs in commonly-used cell lines with minimal resource coupling have been identified, a more general solution to transcriptional resource competition would enable the use of any combination of promoters and TAs in any cell line. A resource-decoupled genetic module comprised of the genetic device of interest and a feedforward controller that makes the genetic device's output insensitive to perturbations in resource availability was then designed, thereby decoupling expression of the module's output from resource usage by other modules (FIGS. 1B-1C). The feedforward controller manifests as an iFFL: an endoRNase protein is expressed from the identical promoter as the output, then binds and cuts a specific target site in the 5′UTR of the output mRNA, leading to its degradation and preventing translation of the output protein. Both transcription units utilize identical promoters in order to couple the transcriptional inputs to both genes; global changes to the concentration of transcriptional inputs including ectopically or endogenously expressed transcription factors (TFs), transcription coactivators and corepressors, and RNA polymerase and its cofactors will affect both genes equally. In addition, since the transcription units are either placed on the same strand of DNA or are delivered to cells in a correlated fashion, the number of each transcription unit is always proportional.

Using a simple mathematical model of the iFFL module with gene expression resources as an input, it was predicted that resource loading would proportionally affect both the endoRNase and the output expression, such that changes in endoRNase levels could offset changes in output production and make the output expression insensitive to resource loading (FIG. 1D). A toolkit was developed for creating post-transcriptional genetic circuits using Cas6- and Cas13-family CRISPR endoRNases. These endoRNases show very strong repression of protein expression (˜50- to 250-fold knockdown) and good orthogonality in terms of targeting specific RNA hairpins (FIG. 2E). To implement our iFFL design, one of the stronger endoRNases in this toolkit, CasE, was utilized. However, this design may be implemented by any of the other endoRNases shown in FIG. 2E, as well as endoRNases from other sources with similar specificity of RNA targeting. The endoRNase and output mRNAs are transcribed as separate RNA species but from identical promoters (P) on the same strand of DNA with no insulator in between, helping to couple their transcriptional inputs and kinetics.

Previous miRNA-based iFFL designs placed miRNA target sites in the 3′UTR of the output genes. However, placing miRNA target sites in the 3′UTR and 5′UTR separately leads to improved gene knockdown. Therefore, the effect of placing the endoRNase target site in either the 5′UTR or the 3′UTR of the output was tested. The result showed much better knockdown and adaptation to DNA copy number when target sites were placed in the 5′UTR (FIG. 2F). Thus, the present design incorporated endoRNase target sites at the 5′UTR of the output molecule coding sequence. The CasE target sites were therefor placed in the 5′UTR because endoRNases more strongly knock down gene expression when target sites are in the 5′UTR rather than the 3′ UTR.

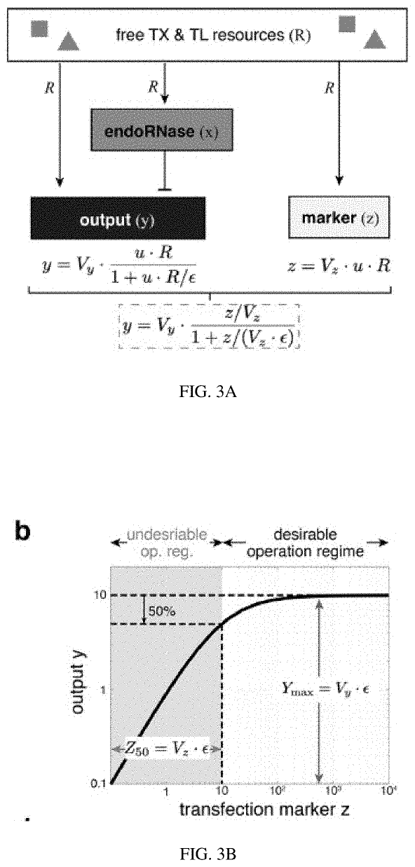

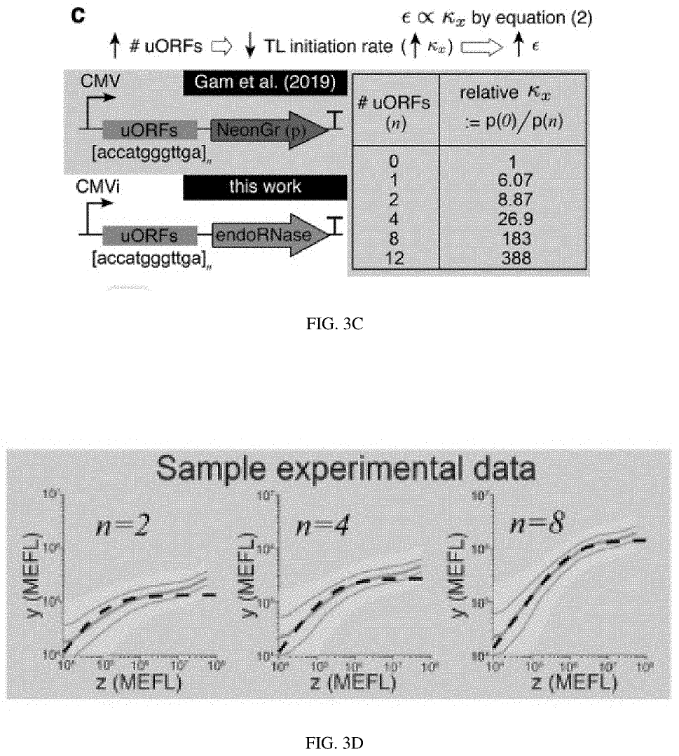

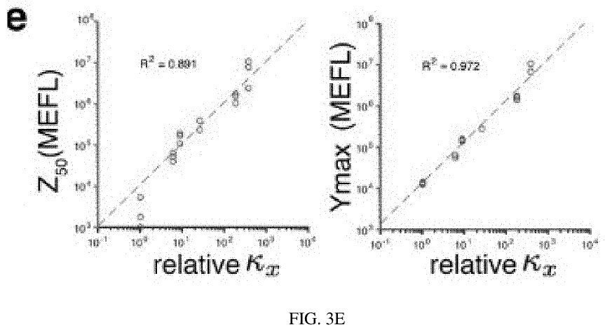

The ability of the iFFL to make output expression level insensitive to resource availability is revealed through a mathematical model of the iFFL. The model also predicts that the robustness of the iFFL-regulated output can be tuned through variable numbers of short upstream open reading frames (uORFs) in the 5′UTR of the endoRNase transcription unit. With reference to FIG. 3A, the iFFL module consists of an endoRNase (x) that targets the mRNA my of the output protein (y) for cleavage. The two proteins are encoded on the same DNA plasmid and driven by identical promoters. This ensures that the two genes share the same pool of transcriptional resources (i.e., CoAs). It appeared that the endoRNase x enzymatically degrades the output's mRNA following Michaelis-Menten kinetics. Under these assumptions, the steady state output protein concentration can be written as:

$$y = \frac{{α}_{y} {β}_{y} uR}{{γ}_{y} {k κ}_{y} {δ}_{y}} \cdot {[ 1 + θ \frac{{α}_{x} {β}_{x} uR}{{γ}_{x} {k κ}_{x} {δ}_{x} {δ}_{y} {K}_{M}} ]}^{- 1} , \qquad ( 1 )$$

where R: =RTX RTL lumps the free concentrations of the transcriptional resource

RTX and the translational resource RTL and u is the concentration of the DNA plasmid that encodes both genes. For i=x, y, parameter αi is the transcription initiation rate constant of gene i; δi is the decay rate constant of the mRNA transcript mi; γi is the decay rate constant of protein i; βi is the translation initiation rate constant, and xi is the dissociation constant describing the binding between translational resource (i.e., ribosome) and the mRNA transcript mi and thus governs translation initiation. The parameter θ is the catalytic rate constant of the endoRNase cleaving my; KM is the Michaelis-Menten constant describing the binding of the endoRNase with my, and k is the dissociation constant describing binding of transcriptional resource with the identical promoters driving the expression of both x and y. To determine how each parameter changes the iFFL module's response to variations in resource availability, the expression of y in (1) was simplified by introducing the following lumped parameters:

$${V}_{y} := \frac{{α}_{y} {β}_{y}}{{γ}_{y} {k κ}_{y} {δ}_{y}} , \\ and \\ ϵ := \frac{{γ}_{x} {k δ}_{x} {δ}_{y} {K}_{M}}{{α}_{x} {β}_{x} θ} \cdot {κ}_{x} , \\ and re - write ( 1 ) as : \qquad ( 2 )$$

$$y = {V}_{y} \cdot \frac{u \cdot R}{1 + u \cdot R / ϵ} . \qquad ( 3 )$$

Note that by (2), for a fixed output gene, the parameter Vy is fixed and does not change with any physical parameter of the endoRNase. On the other hand, changing the physical parameters governing the production, decay, and enzymatic reactions of the endoRNase only changes the lumped parameter ε. According to (3), for u·R/ε >>1, the equation is y≈Ymax:=Vy·ε, which is independent of R, and therefore independent of the free concentrations of both transcriptional and translational resources. This implies that if the parameter ε was designed to be sufficiently small, the iFFL module's output can adapt to variations in resource availability.

To experimentally quantify the iFFL module's robustness to resource availability, the fluorescence level of a co-transfected transfection marker (TX Marker) protein z was used as a proxy for the free amount of resources R. This is because the steady state of z can be written as z=Vz·u·R, where Vz is a lumped parameter independent of u and R and defined similarly to Vy in (2). This enables us to re-write y in (3) as a function of the experimentally measurable quantity z:

$$y = {V}_{y} \cdot \frac{z / {V}_{z}}{1 + z / ( {V}_{z} ϵ )} . \qquad ( 4 )$$

An experimentally quantifiable inverse measure of robustness, Z50, which is the TX Marker's fluorescence level at which the iFFL module's output is half of its maximum value, was then introduced (FIG. 3B) (i.e. y≥Ymax/2 for all z≥Z50). By substituting y=Ymax/2 into equation (3), it was found that Z50=Vzε, implying that robustness increases as the parameter ε decreases.

A library of resource-decoupled device modules with different ε parameters was therefore constructed. To construct this library, the number of uROFs (n) in the 5′UTR of the endoRNase's transcript mx was increased to effectively increase the dissociation constant κx between the ribosome and mx37, thus increasing ε. With reference to FIG. 3C, the relationship between n and κx was experimentally characterized in 38, where the authors measured expression of a constitutive fluorescent protein p with different numbers of uORFs in the 5′UTR of its transcript. Since the expression level of a constitutive gene is inversely proportional to the dissociation constant between ribosome and its transcript (i.e., p∝1/κx, see), the equation is:

$$relative {κ}_{x} = ( relative {κ}_{x} ) ( n ) := \frac{{κ}_{x} ( n )}{{κ}_{x} ( 0 )} = \frac{p ( 0 )}{p ( n )} , \qquad ( 5 )$$

where p(n) and κx(n) are the steady state expression of p and the dissociation constant between ribosome and protein p's mRNA transcript in the presence of n uORFs, respectively. Since it has been derived from equation (4) that (i) Ymax and Z50 are both proportional to ε and hence proportional to κx and that (ii) κx(n)=(relative κx)(n)×κx(0) according to (5), our model predicts that Ymax =Ymax(n) and Z50=Z50(n) are both proportional to relative κx.

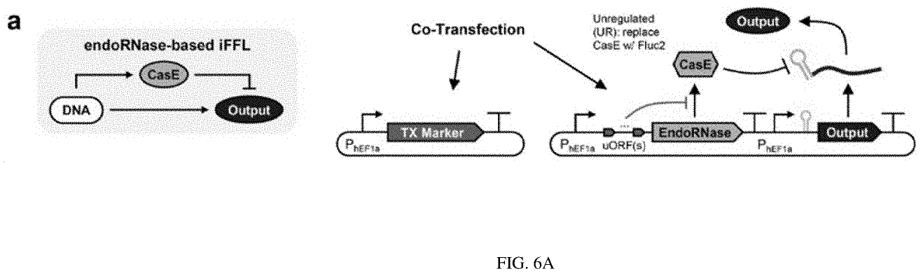

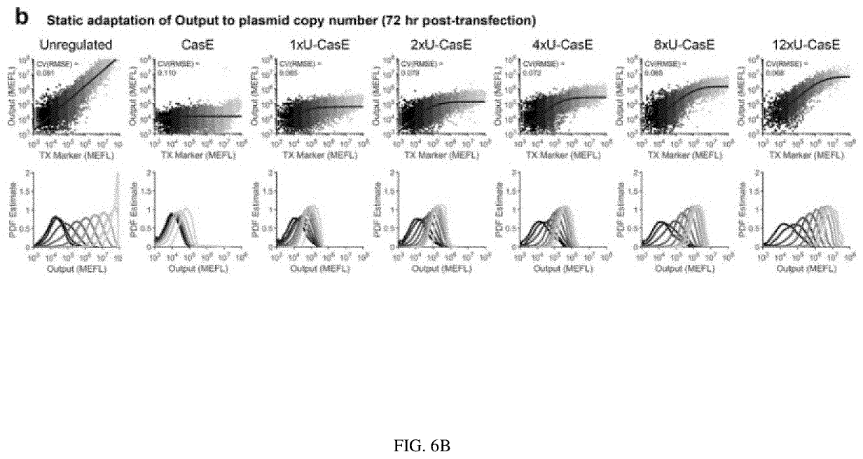

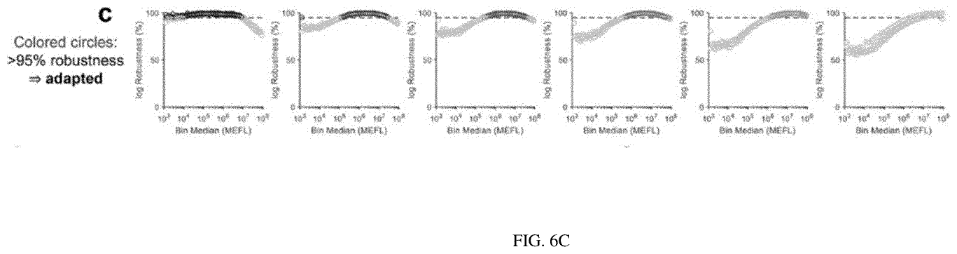

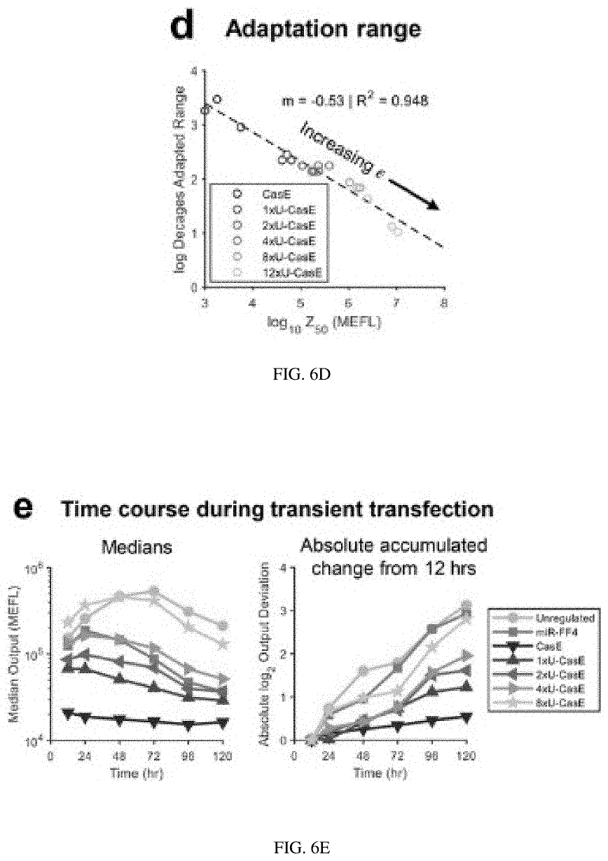

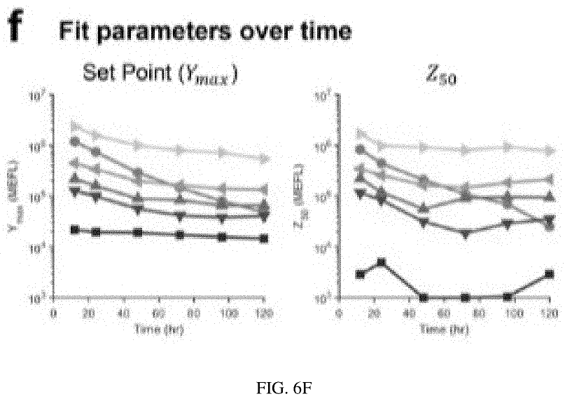

To verify this model prediction, for n=0, 1, 2, 4, 8 and 12, the iFFL modules' output (y) for different levels of TX Marker (z) was plotted. The shape of the experimentally measured TX Marker vs output dose response curves (see FIG. 3D for select samples and FIG. 6D for all data) matches well with the model prediction in FIG. 3B, suggesting that Z50 is a reasonable inverse measure of the module's robustness. The experimental data with was fit with (4) and evaluate the fitting function to describe Ymax and Z50 for different n in the experimental data. In FIG. 3E, Ymax and Z50 were plotted against the relative κx values listed in FIG. 3C. It was observed that Ymax and Z50 are both linearly related to relative κx, indicating that our model (4) can capture the salient steady state behavior of the iFFL module. For a fixed output gene (i.e. given Vy), since Z50 and Ymax only depend on ε according to (3), our model also highlights a key design trade-off for an iFFL module: increasing maximum output Ymax via tuning εnecessarily increases Z50, which indicates a decrease in robustness. The number of uORFs on endoRNase's transcript can thus serve as a convenient knob to balance this trade-off between robustness and maximum expression level. To increase Ymax without affecting Z50, the relative promoter copy number of the output can be increased relative to the endoRNase, as was demonstrate with poly-transfection.

In addition to robustness to variations in free transcriptional and translational resource concentrations, the iFFL can also attenuate the effect of DNA plasmid variation (i.e. changes in u) on the module's output. In fact, since u and R are clustered together in (3), our analysis on the module's robustness to R carries over directly when analyzing its robustness to u: when uR>>ε, it would be y≈Vyε according to (3), which is independent of u. Robustness to variations in u also includes temporal variability of DNA concentration, which is present in transient transfection experiments due to dilution of DNA plasmids as cells grow and divide. As one decreases the number of uORFs in the endoRNase's transcript, our model predicts that the iFFL module becomes more robust to DNA copy number variability in the sense that it's output remains the same for a wider range of DNA copy numbers (i.e. smaller Z50). This allows the module's output to maintain Ymax for a longer period of time as DNA concentration gradually decreases, a phenomenon that was observed both experimentally and numerically.

### The Resource-Decoupled Module's Output is Robust to Resource Loading by Gal4 TAs

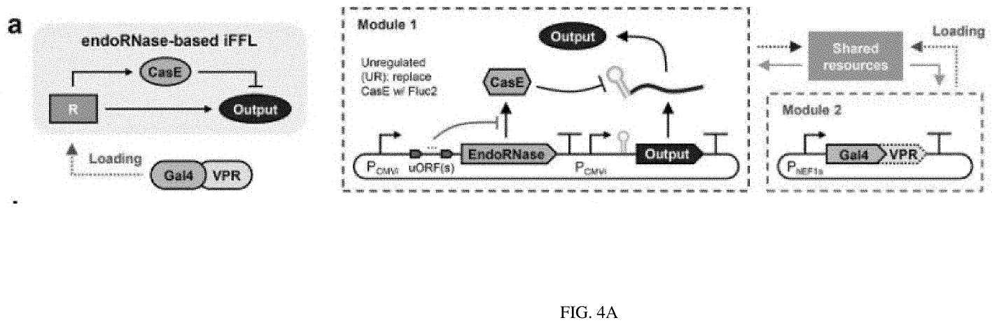

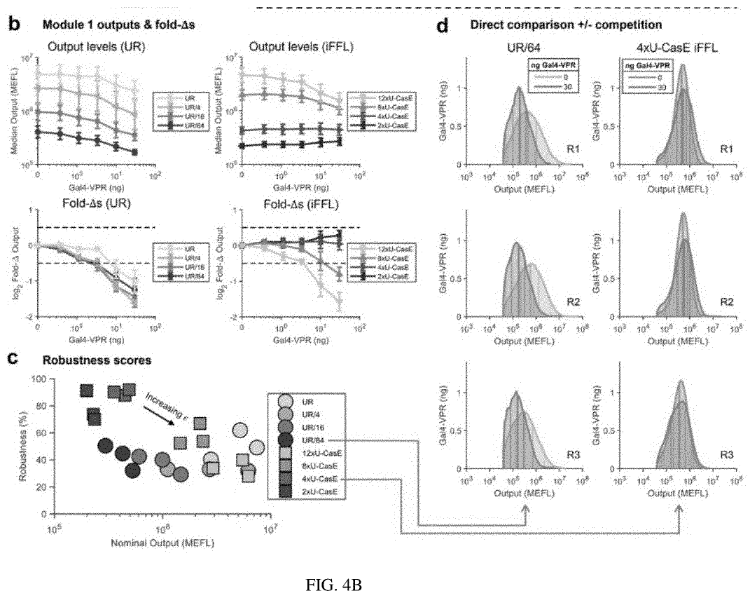

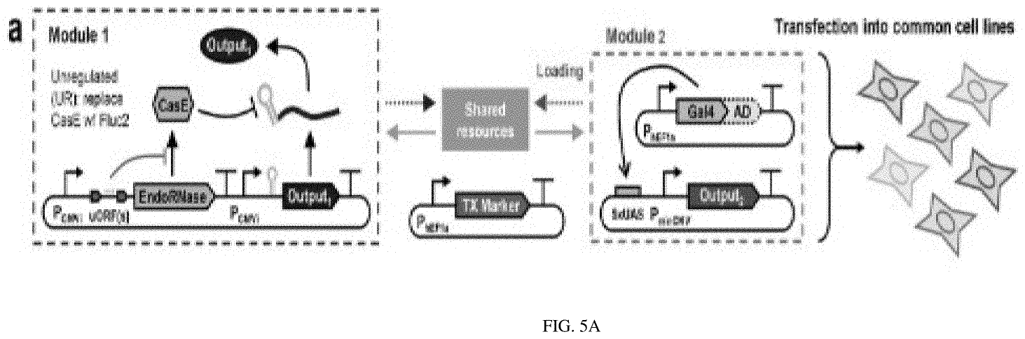

To determine the extent to which the output expression of the iFFL design is insensitive to resource loading, the CasE-based iFFL was constructed as shown in FIG. 4A, which uses the CMVi promoter to drive expression of both CasE and the output. the CMVi promoter was selected because of all the combinations of activators, promoters, and cell lines tested, the largest fold-decrease in promoter expression was caused by Ga14-VPR on CMVi in HEK-293FT cells (FIGS. 2A-2D). The CMVi iFFL plasmid was transfected along with plasmids bearing a hEF1 α-driven transfection marker and hEF1a: Gal4-VPR into HEK-293FT cells to measure the response of the iFFL to resource loading. 0, 1, 2, 4, 8, or 12 uORFs (5′-ACCATGGGTTGA-3′) were placed in front of CasE to reduce its translation rate by between 2- and 200-fold and thereby proportionally tune key ε parameter. As a control, an unregulated (UR) variant of the iFFL module which replaced CasE with the luminescent protein Fluc2 was made, and thus did not form an iFFL.

To account for differences in protein expression levels between the UR and iFFL modules, cells were transfected with equimolar, 1:4, 1:16, or 1:64 dilutions of the UR plasmid relative to the iFFL plasmid in samples transfected with iFFL variants. To quantify the degree to which an iFFL or UR module is sensitive to resource loading by Gal4-VPR, the fold-changes relative to nominal output was measured (i.e. the median output in the absence of Gal4-VPR) and from those computed robustness scores:

$$Fold - Δ ( Gal 4 - VPR = x ) ) = \frac{Output ( Gal 4 - VPR = x )}{Output ( Gal 4 - VPR = 0 )}$$

Robustness(Gal4-VPR=x)=100 (1−|1″Fold-Δ(Gal4-VPR=x)|);

Our results from co-transfecting the iFFL and UR plasmids with increasing amounts of Gal4-VPR show that variants of the iFFL with 4 or fewer uORFs in front of CasE are significantly less affected by Gal4-VPR than the UR controls (FIG. 4B). At the highest dosage of Gal4-VPR tested (30 ng), the output of the UR samples decreased between 2- and 3-fold, whereas the iFFL variants with 4× or 2× uORFs changed by less than 1.5-fold (FIG. 4B). In terms of robustness, most UR samples ranged between 30% and 60% regardless of the nominal output level. The iFFL samples with lower nominal output (higher CasE levels obtained via fewer uORFs) showed high robustness (70-90%), as predicted from the model due to a lower ε. iFFL samples with higher nominal output (lower CasE level obtained via more uORFs) showed lower robustness also as expected by the model due to a larger ε. The samples with 12x-uORFs show comparable robustness to the UR samples. Direct comparisons of the full output distributions of UR and iFFL samples with comparable nominal outputs, in the presence and absence of Gal4-VPR, are shown in FIG. 4B (distributions are shown for each experimental repeat). From these histograms, it was observed that Gal4-VPR competition causes the entire distributions of the UR sample (UR 1:64 diluted) to shift down in expression, whereas the distributions of the iFFL sample (4× uORFs) retained approximately the same median with comparable variance. Taken together, this data validates the design demonstrating increased robustness for the iFFL module and that as predicted from theory, this robustness can be tuned by tuning the parameter ε with uORFs.

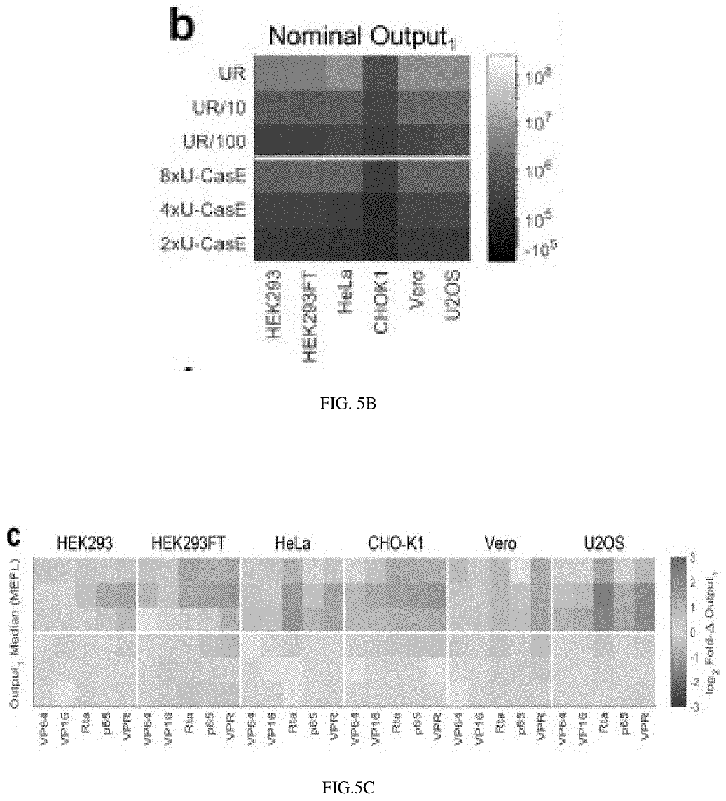

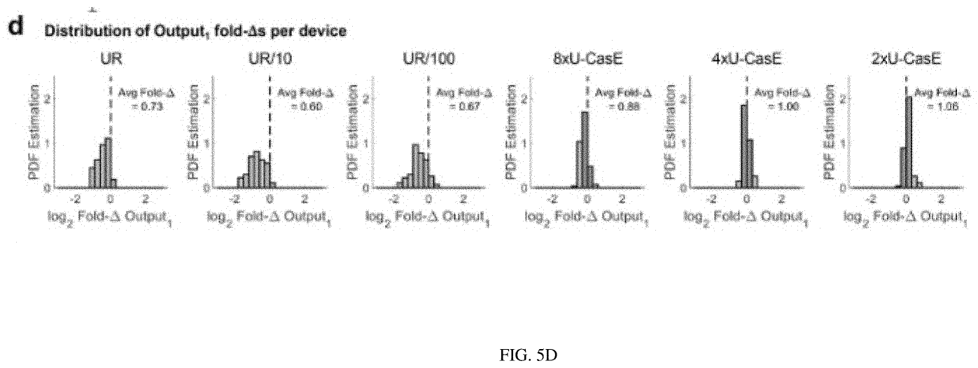

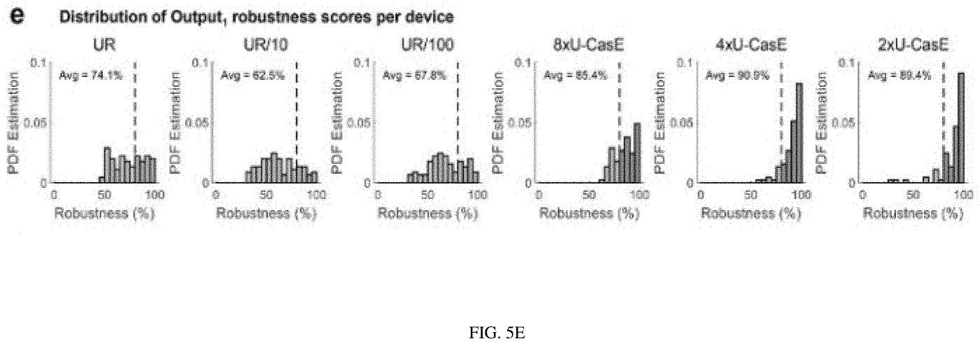

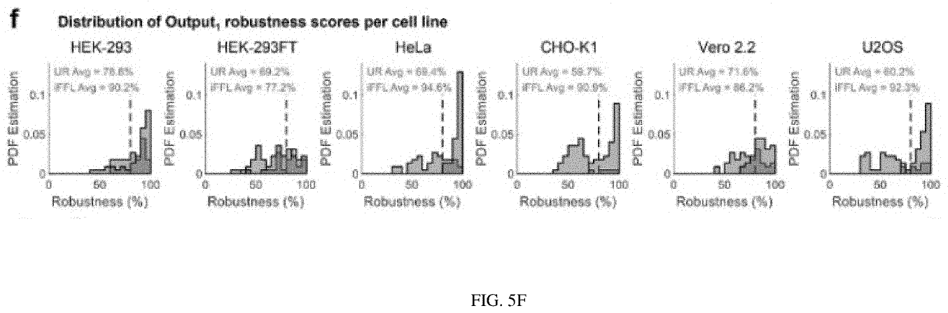

Next, whether the iFFL module functions in other cell lines and whether its output expression is insensitive to resource loading by different Gal4 TAs (FIG. 5A) was tested. Overall, it was found that the fold-changes in CMVi iFFL output in response to resource competition are much lower than the UR controls for all Gal4 TAs and cell lines tested (FIGS. 5B-5C). On average, the UR variants were knocked down between 30% and 40% whereas the iFFL variants changed by 15% or less (FIG. 5D). As with the experiment shown in FIGS. 4A-4B, it was observed that larger fold-changes in iFFL variants with 8 uORFs compared to those with fewer. The robustness scores of UR-activator combinations across cell lines were as low as ˜30% and less than a third (30.7%) of the combinations had robustness above 80% (FIG. 5E). By contrast, the iFFL variants had robustness above 80% for the large majority of combinations (81.5%). On average, the robustness scores of the UR variants ranged between 60% and 75%, whereas those of the iFFL variants ranged between 85% and 90%.

The distribution of robustness scores per cell line for all iFFL and UR variants, as well as constitutive promoters from FIGS. 2A-2D, are compared in FIG. 5F. It was found that the iFFLs variants showed the highest robustness in HeLa and U2OS cells (100% and 93.3% of combinations over 80% robustness averaging 94.6% and 92.3%, respectively). The high robustness of the iFFL in CHO-K1 cells (84.4% above 80%, averaging 90.9%) is particularly striking in comparison to both the low robustness of the UR variants in that cell line (8.90% above 80%, averaging 59.7%) and our earlier observation that nearly all combinations of constitutive promoters and activators tested in CHO-K1 cells led to decreased expression (FIGS. 2A-2D). The iFFL variants in HEK-293FT cells showed relatively low robustness (46.7% above 80%, averaging 77.2%) compared to other cell lines, though still higher than the UR variants (37.8% above 80%, averaging 69.2%). This relatively poor performance in HEK-293FT cells appears to result from a slight increase in expression of the iFFL output in response to the Gal4 TAs. Overall, these results demonstrate that the iFFL output is robust to resource loading by different resource competitors across varying cell line contexts, suggesting it is a general solution easily applicable across cell types.

To ensure that our results were not specific to the CMVi promoter, the experiments in FIGS. 4A-4B & FIGS. 5A-5E were repeated with a version of the iFFL which replaces the CMVi promoters with hEF1a. As with the CMVi iFFL, variants of the hEF1a iFFL with fewer uORFs (smaller ε) showed reduced fold-changes and higher robustness scores in response to Gal4 TAs than UR variants with comparable nominal outputs. Compared to the CMVi iFFL, the hEF1a iFFL generally showed higher fold-changes and lower robustness scores, especially in U2OS and HeLa cells co-transfected with Gal4-Rta. Interestingly, the hEF1a iFFL output for variants with 4 or fewer uORFs was slightly increased (<1.5-fold) by the Gal4 TAs in HEK-293 and HEK-293FT cells. This increase apparently results from toxicity by the Gal4 TAs being exacerbated by the use of several hEF1a promoters in the circuit. Toxicity may reduce the growth rate of the cells and thus alter the circuit's dynamics. In support of this notion, transfecting the hEF1a iFFL variants into HEK-293FT cells with a less toxic reagent Viafect (rather than Lipofectamine 3000) eliminates Gal4-VPR-dependent toxicity as well as the increase in output expression. Overall, like the CMVi iFFL, the hEF1a iFFL module is much more robust than comparable UR variants to resource loading by Gal4 TAs.

### The Resource-Decoupled Module Output Adapts to Plasmid DNA Copy Number Variation

From the model of our endoRNase-based iFFL design, when z/ε >>1, the iFFL output will also be robust to variation in the DNA copy number of the iFFL. Whether the output expression of the hEF1a iFFL could adapt to the multiple log decades of variation in plasmid uptake between individual cells seen in transient transfections was tested (FIG. 6A). uORFs ertr used to tune ε, smaller values of which are predicted by the model to make iFFL output adapt to larger ranges of DNA copy numbers. Fits of the 2D transfection marker vs iFFL output curves given by equation (4) (FIGS. 3A-3B) are shown overlaid on the cell scatterplots in FIG. 6B. For the UR samples, the output is proportional to the transfection marker, so it fit with a simple linear formula: Output=m·TX Marker. The histograms below the scatterplots illustrate that the distribution of expression for cells in different transfection marker-delineated bins is unaffected by changes in DNA copy number over multiple log decades of copy number variation. Similar bin histograms for UR outputs diluted 1:1, 1:4, 1:16, or 1:64 compared to the iFFL output plasmid show that decreasing expression of the output does not itself cause adaptation to DNA copy number.

To quantify the extent of adaptation of iFFL output expression to DNA copy number, the median expression of cells was compared in finely-sampled transfection marker-delineated bins to the fit value of Ymax, and considered a bin to be ‘adapted’ to copy number variation if log10 (output) was within 5% of log 10 (Ymax) (i.e. the log-scale robustness score was above 95%-FIG. 6C). As expected based on the model, increasing ε by increasing the number of uORFs decreases the range over which the iFFL output adapts to DNA copy number (FIG. 6d). Since Ymax is also correlated to ε, fit values of Ymax are also highly correlated with the adaptation range. These experiments were repeated and analysed with the CMVi-driven CasE iFFL and similar results were found.

Since the iFFL can adapt to DNA copy number variations between cells, whether the iFFL can also adapt to DNA copy number variations within a single cell were investigated. In particular, iFFL output was measured over time following transient transfection, during which the dynamics of plasmid DNA expressing the iFFL module are characterized by a step-increase followed by dilution due to cell division. Thus, the dynamics of any constitutively-expressed gene transiently transfected into dividing cells resembles a pulse. According to our modeling results, the iFFL output should be robust to slow temporal changes of DNA copy number. Indeed, variants of the iFFL with fewer uORFs (and thus smaller ε) exhibited smaller changes in median expression over the time period of 120 hours post-transfection compared to a UR control as well as a miRNA-based iFFL (FIG. 6E). The miRNA-based iFFL used miR-FF4 and was similar to previous designs, with the miR-FF4-expressing intron moved to the 3′UTR and the miRNA target site moved to the 5′UTR. In both the CasE and miR-FF4 iFFLs, placing the target sites for either FF4 or CasE in the 5′UTR rather than the 3′UTR significantly improves adaption to DNA copy number. During the time courses, the CasE iFFL showed less change over time than the miR-FF4 iFFL: at 120 hours, the miR-FF4 and 2x-uORFs CasE iFFLs both had similar median output levels, but the maximum miR-FF4 iFFL output was ˜2-fold higher (FIG. 6E). Finally, it was observed that the fit parameters Ymax and Z50 of the CasE iFFL variants remain largely unchanged between 48 and 120 hours, whereas those of the miR-FF4 iFFL decreased 5-10-fold over the same period (FIG. 6F). Overall, these data demonstrate that the CasE iFFL can also accurately set gene expression levels regardless of DNA dosage to cells and in the face of dynamic transcriptional disturbances such as plasmid dilution.

### Discussion

The development of sophisticated synthetic genetic circuits will be enabled through improved understanding of how the function of a genetic component is affected by its context. Competition between genes and their products for shared gene expression resources is an important factor causing context-dependence. Several biomolecules at all levels of gene expression in mammalian cells are known to be shared among many genes and known to induce couplings in their expression. However, the effects of competition for these resources on engineered genetic systems has not been systematically investigated. The focus of the present disclosure was thus on understanding the extent to which competition between genetic devices for shared transcriptional resources affects gene expression in synthetic genetic systems. While improved characterization of resource competition effects can improve genetic circuit design9, it has also been shown that gene expression controllers can be used to automatically mitigate the effects of resource competition in bacterial systems. To provide a solution to the resource competition problem in mammalian cells, a simple genetic controller that can be added to any genetic device was developed to make its expression level robust to resource availability. This iFFL effectively makes gene expression robust to resource loading by different Gal4 TAs across cell lines, demonstrating the applicability of the system to variable transcriptional contexts.

From our characterization of resource competition, it was found that constitutive promoters are affected disparately by different Gal4 TAs (FIGS. 2A-2D). The differences in promoter responses may result from the promoters utilizing different subsets of transcriptional resources40: there are hundreds of transcriptional cofactors (including CoAs and subunits of the mediator complex) that interact with native and synthetic TFs. It was recently shown that TATA-box based and CpG island-based core promoters are activated by different subsets of CoAs43. Consistent with these results, it was found that the responses of promoters with large CpG islands to Gal4 TAs were more similar to each other than to promoters without CpG islands.

In each cell line, several combinations of promoters and Gal4 TAs for which the TA minimally affected promoter expression were identified. These ‘non-coupled’ combinations may result from the TA recruiting different specific CoAs than those used by the constitutive promoter, and/or from the promoter having relatively high affinity for CoAs. The combinations with minimal or reduced coupling will be useful for choosing parts in synthetic genetic circuits that, when combined together, enable more accurate prediction of circuit behavior. Additionally, relatively strong constitutive promoters that are less affected by resource loading may be utilized as more reliable transfection markers.

A previous study called into question whether squelching by TAs was an artifact that only affected episomal genes by showing that self-squelching only occurs if the TA-driven promoter is in a plasmid, but not in the genome. However, other experiments have demonstrated coupling between signal-responsive genes in the genome and there is growing evidence for the role of squelching in natural gene regulation. Our results show that both non-target squelching and self-squelching can indeed affect integrated genes. Thus, our results are extendable to various contexts, including episomal and genomic genetic systems.

In various cell lines and in combination with different Gal4 TAs, the results herein showed that our endoRNase-based iFFL design can effectively cancel-out the effects of transcriptional resource competition on gene expression output (FIGS. 4A-5F). From the model of the the iFFL design, a parameter inversely proportional to the robustness of the iFFL, ε, was identified. ε was tuned by placing variable numbers of uORFs in the 5′UTR of the iFFL endoRNase (CasE). The experiments validated the model prediction that smaller values of ε lead to higher robustness of the iFFL output level to resource competition (FIGS. 4A-5F) and a larger range over which the iFFL output adapts to DNA copy numbers (FIG. 6A-6F). In the iFFL design, tuning ε also affects the iFFL's output expression level, yielding an inherent trade-off between the set-point of the iFFL and its robustness to transcriptional perturbations. The output level can be independently tuned from the robustness via the parameter Vy, which is proportional to the transcription, translation, mRNA degradation, and protein degradation rates of the output protein. To validate this relationship, poly-transfection was used to sample the iFFL at various ratios of CasE and the output plasmids, finding that indeed, increasing the output DNA dosage (and thus transcription rate) relative to that of CasE enabled higher Ymax at equivalent values of Z50. Tuning the relative gene dosage is not possible in all genetic systems and tuning relative transcription rates by changing promoters may break the coupling of resource inputs between the output and endoRNase transcription units. More generally, RNA aptazymes46 and inducible protein degradation domains could be utilized to independently tune RNA or protein stability of the output without affecting the ε parameter.

Notably, it was found that the nominal output levels (median expression in the absence of resource competition) and fit Ymax parameters for both the CMVi and hEF 1 a iFFLs were highly correlated across cell lines. To a simple approximation, differences in gene expression between cell lines can be attributed to the differences in gene expression resources in the cells. If this approximation is valid, then the model of our iFFL (see Section 2.3) predicts that the iFFL output expression will be similar in different cell lines. Indeed, the median output and Ymax values in HEK-293FT cells well-predicted those in other cell lines, with R2>0.8 in all cases except for the CMVi iFFL in CHO-K1 cells, which had both values consistently ˜4-fold lower than those in HEK-293FT cells. Overall, the iFFL/uORF strategy of setting and tuning gene expression levels enables much higher cell-to-cell consistency in expression levels than using unregulated devices or different constitutive promoters of varying strength.

Many existing genetic devices are easily amenable to augmentation by our iFFL design. Augmentation can be achieved by adding (i) an endoRNase driven by the identical promoter as the output of the original device and (ii) an endoRNase target site in the 5′UTR of the device's output mRNA (FIG. 1). In bacterial genetic circuits, the endoRNase target site can instead be placed in-frame between the RBS and coding sequence, as previously shown with Cys450. It is feasible that many such iFFLs can be created in a single cell to independently control expression of different genes. Our iFFL may be useful in many applications, including controlling the ratios of genetic device components to maximize device performance38, making dCas9-based circuits robust to shared dCas9 resources, and precisely setting the levels of signaling receptors to achieve unique input functions.

To our knowledge, this was the first iFFLs that can be used to mitigate the effects of resource competition. For over a decade it has been known that the iFFL topology may enable a genetic device's output to adapt to perturbations54. This property have been exploited to create iFFLs that can adapt to DNA copy number variation or inducer input levels. Previous solutions to the ribosome competition problem in bacteria utilized negative feedback loops (NFBLs). An advantage of NFBLs is that they can tolerate unknown dynamics in the output gene expression process, whereas iFFLs require the effect of the disturbance to exactly be canceled out by the controller species. Conversely, iFFLs are generally much simpler to design and operate. Recent work showed that combining miRNA-based iFFLs and NFBLs yielded circuits with some properties of both control mechanisms, indicating that iFFLs and NFBLs are not mutually exclusive and can synergize when used together.

In bacteria where competition for ribosomes is most prominent8, it has been proposed that centralized controllers for ribosome levels can ensure a constant supply regardless of loads placed on the ribosome. Conversely, the number of CoAs and GTFs competed for by eukaryotic TAs and promoters is likely far too large to build such a centralized controller. Even if the mediator complex were the only shared transcriptional resource, it is comprised of dozens of domains, each of which would need to be controlled.

In addition to resource loading, It was found that our iFFL design is also robust to static and dynamic variability in its DNA copy number (FIGS. 6A-6F). Our endoRNase-based iFFL output adapted to copy number variation over ˜1-2 log decades, depending on the value of ε. This range of adaptation is comparable to the TALER-based iFFL implemented by Segall-Shapiro et al. in bacteria30 and is a major improvement compared to the current standard of miRNA-based iFFLs in mammalian cells. EndoRNase-based iFFLs have several advantages over miRNA-based designs, including independence from accessory factors such as RISC, the ability to make gene expression robust to changes in translational resources, and apparently faster dynamics.

Measurements of the CasE iFFL output dynamics during transient transfection showed improved stability over time compared to a UR device and a miRNA-based iFFL (FIGS. 6A-6F). Simulations with an ODE model of the CasE iFFL indicated that changes in iFFL output over time during a transient transfection can be minimized by making the dynamics of CasE expression fast compared to cell division. The median degradation rate of native miRNAs (half-life ˜20 hours) is comparable to the typical rate of cell division for a mammalian cell (doubling time ˜20 hours). Because the CasE iFFL output varies much less than the miR-FF4 iFFL output during transient transfection, our modeling thus suggests that the CasE protein may have a relatively fast degradation rate compared to miR-FF4 and cell division. This data and modeling points to an interesting new mechanism to control the temporal dynamics of iFFLs.

Overall, presented herein are characterization of transcriptional resource competition in mammalian cells and the design and performance testing of an endoRNase-based iFFL design which mitigates the effects of resource competition on gene expression. Our characterization of resource competition will be useful both for designing genetic circuits with minimal competition among genetic devices composing the circuits, as well as for predicting the behavior of complex circuits composed of genetic devices which compete for shared resources. Our iFFL is a simple and accurate controller of gene expression that will find many uses in engineering mammalian cells. Altogether, this work will enable more accurate bottom-up design of genetic systems in mammalian cells, facilitating the development of more complex and reliable circuits for applications in cell therapy, tissue/organoid engineering, and cellular bioproduction.

### Methods

### Modular Plasmid Cloning Scheme

Plasmids were constructed using a modular Golden Gate strategy similar to previous work in our lab. Briefly, basic parts (insulators, promoters, 5′UTRs, coding sequences, 3′UTRs, and terminators—termed level 0s (pL0s)) were assembled into transcription units (TUs—termed level 1s (pL1s)) using BsaI Golden Gate reactions. TUs were assembled into multi-TU plasmids using SapI Golden Gate reactions. To make lentivirus transfer plasmids, pL0s or pL1s were cloned into a vector derived from pFUGW (AddGene plasmid #14883) using either BsaI or SapI Golden Gate, respectively.

### Cell Culture

HEK-293 cells (ATCC), HEK-293FT cells (Thermo Fisher), HeLa cells (ATCC), and Vero 2.2 cells (Massachusetts General Hospital) were maintained in Dulbecco's modified Eagle media (DMEM) containing 4.5 g/L glucose, L-glutamine, and sodium pyruvate (Corning) supplemented with 10% fetal bovine serum (FBS, from VWR). CHO-K1 cells (ATCC) were grown in F12-K media containing 2 mM L-glutamine and 1500 mn/L sodium bicarbonate (ATCC) supplemented with 10% FBS. U2OS cells (ATCC) were grown in McCoy's 5A media with high glucose, L-glutamine, and bacto-peptone (Gibco) supplemented with 10% FBS. All cell lines used in the study were grown in a humidified incubator at 37 deg and 5% CO2. All cell lines tested negative for mycoplasma.

### Transfections

Cells were cultured to 90% confluency on the day of transfection, trypsinized, and added to new plates simultaneously with the addition of plasmid-transfection reagent mixtures (reverse transfection). Transfections were performed in 24-well or 96-well pre-treated tissue culture plates (Costar). Following are the volumes, number of cells, and concentrations of reagents used for 96-well transfections; for 24-well transfections, all values were scaled up by a factor of 5. 120 ng total DNA was diluted into 10 μL Opti-MEM (Gibco) and lightly vortexed. The transfection regent was then added and samples were lightly vortexed again. The DNA-reagent mixtures were incubated for 10-30 minutes while cells were trypsinized and counted.

After depositing the transfection mixtures into appropriate wells, 40,000 HEK-293, 40,000 HEK-293FT, 10,000 HeLa, 20,000 CHO-K1, 20,000 Vero 2.2, or 10,000 U2OS cells suspended in 100 μL media were added. Lipofectamine LTX (ThermoFischer) was used at a ratio of 1 μL PLUS reagent and 4 μL LTX per 1 μg DNA. PEI MAX (Polysciences VWR) was used at a ratio of 3 μL PEI per 1 μg DNA. Viafect (Promega) was used at a ratio of 3 μL Viafect per 1 μg DNA. Lipofectamine 3000 was used at a ratio of 2 μL P3000 and 2 μL Lipo 300 per 1 μg DNA. Attractene (Qiagen) was used at a ratio of 5 μL Attractene per 1 μg DNA. For experiments with measurement windows between 12-72 hours (as indicated on the FIGs or in their captions), the media of the transfected cells was not replaced between trasnfection and data collection. For experiments with measurements at longer time points, the transfected cells were passaged at 72 hours in fresh media on a new plate. In order to maintain a similar number of cells for data collection at longer time points, transfected cells were split at ratios of 1:2 or 1:4 for samples being collected at 96 or 120 hours, respectively. For all transfections with Doxycycline (Dox, Sigma-Aldrich), Dox was added immediately after transfection unless otherwise indicated.

In each transfection sample, a hEF1a-driven transfection marker was included to indicate the dosage of DNA delivered to each cell and to facilitate consistent gating of transfected cells. Of the strong promoters tested (CMV, CMVi, and hEF1a), the hEF1a promoter gave the most consistent expression across cell lines and was generally less affected by resource loading by Gal4 TAs.

### Lentivirus Production and Infection

Lentivirus production was performed using HEK-293FT cells and second-generation helper plasmids MD2.G (Addgene plasmid #12259) and psPax2 (Addgene plasmid #12260). HEK-293FT cells were grown to 90% confluency, trypsinized, and added to new pre-treated 10 cm tissue culture plates (Falcon) simultaneously with addition of plasmid-transfection reagent mixtures. Four hours before transfection, the media on the HEK-293FT cells was replaced. To make the mixtures, first 3 μg psPax2, 3 μg pMD2.g, and 6 μg of the transfer vector were diluted into 600 μL Opti-MEM and lightly vortexed. 72 μL of FuGENE6 (Promega) was then added and the solution was lightly vortexed again. The DNA-FuGENE mixtures were incubated for 30 minutes while cells were trypsinized and counted. After depositing the transfection mixtures into appropriate plates, 1×106 HEK-293FT cells suspended in 10 mL media were added. 16 hours after transfection, the media was replaced. 48 hours after transfection, the supernatant was collected and filtered through a 0.45 PES filter (VWR). HEK-293FT cells were grown to 90% confluency, trypsinized, and 1×106 cells were resuspended in 2 mL of viral supernatant and together added to a pre-treated 6-well tissue culture plate (Costar). To facilitate viral uptake, polybrene (Millipore-Sigma) was added to a final concentration of 8 m/mL. Cells infected by lentiviruses were expanded and cultured for at least two weeks before use in experiments using the conditions for culturing HEK-293FT cells described above.

### Flow Cytometry

To prepare samples in 96-well plates for flow cytometry, the following process was followed: media was aspirated, 50 μL PBS (Corning) was added to wash the cells and remove FBS, the PBS was aspirated, and 40 μL Trypsin-EDTA (Corning) was added. The cells incubated for 5-10 minutes at 37 deg C. to allow for detachment and separation. Following incubation, 80 μL of DMEM without phenol red (Gibco) with 10% FBS was added to inactivate the trypsin. Cells were thoroughly mixed to separate and suspend individual cells. The plate(s) were then spun down at 400×g for 4 minutes, and the leftover media was aspirated. Cells were resuspended in 170 μL of PBS supplemented with 1% BSA (Thermo Fisher), 5 mM EDTA (VWR), and 0.1% sodium azide (Sigma-Aldrich) to prevent clumping. For prepping larger plates, all volumes were scaled up in proportion to surface area and samples were transferred to 5 mL polystyrene FACS tubes (Falcon) after trypsinization. For standard co-transfections, 10,000-50,000 cells were collected per sample. For the poly-transfection experiment and transfections into cells harboring an existing lentiviral integration, 100,000-200,000 cells were collected per sample.

For the experiments shown in FIGS. 1A-1F 1, samples were collected on a BD LSR II cytometer equipped with a 405 nm laser with 450/50 nm filter (‘Pacific Blue’) for measuring TagBFP or EBFP2, 488 laser with 515/20 filter (‘FITC’) for measuring EYFP or mNeonGreen, 561 nm laser with 582/42 nm filter (‘PE’) or 610/20 nm filter (‘PE-Texas Red’) for measuring mKate2 or mKO2, and 640 laser with 780/60 nm filter (‘APC-Cy7’) for measuring iRFP720. For all other experiments, samples were collected on a BD LSR Fortessa equipped with a 405 nm laser with 450/50 nm filter (‘Pacific Blue’) for measuring TagBFP or EBFP2, 488 laser with 530/30 filter (‘FITC’) for measuring EYFP or mNeonGreen, 561 nm laser with 582/15 nm filter (‘PE’) or 610/20 nm filter (‘PE-Texas Red’) for measuring mKate2 or mKO2, and 640 laser with 780/60 nm filter (‘APC-Cy7’) for measuring iRFP720. 500-2000 events/s were collected either in tubes via the collection port or in 96-well plates via the high-throughput sampler (HTS). All events were recorded and compensation was not applied until data processing (see below).

### Flow Cytometry Data Analysis

Analysis of flow cytometry data was performed using our MATLAB-based flow cytometry analysis pipeline (https://github.com/Weiss-Lab/MATLAB_Flow_Analysis). Arbitrary fluorescence units were converted to standardized molecules of equivalent fluorescein (MEFL) units using RCP-30-5A beads (Spherotech) and the TASBE pipeline process58. Briefly, fluorescence compensation was performed by subtracting autofluorescence (computed from wild-type cells), computing linear fits between channels in single-color transfected cells, then using the fit slopes as matrix coefficients for matrix-based signal de-convolution. Single cells were isolated by drawing morphological gates based on cellular side-scatter and forward-scatter. Threshold gates were manually drawn for each channel based on the fluorescence of untransfected cells. Generally, transfected cells within a population were gated by selecting cells that pass either the gate for the output of interest (Output+) or pass the gate for the transfection marker (TX Marker+).

In FIGS. 2A-2D, the library of constitutive promoters showed different nominal expression levels and were variably affected by competition. Additionally, in order to limit the bias in the reporting of minimally-affected promoters by the the proximity of {P}: Output1 expression to autofluorescence, the analysis of just this dataset incorporates an additional autofluorescence subtraction step.

When first analyzing the data in FIGS. 4A-4B, it was found that the measurements of fold-changes and robustness for the UR variants with diluted output plasmid DNA were sensitive to the fluorescent gating strategy used in the analysis. The typical gating routine of selecting cells positive for either the output or the transfection marker yielded fold-changes of the diluted UR variants that were much larger than when gating on cells positive for just the output. Conversely, both gating strategies yielded similar fold-changes for the iFFL variants regardless of their nominal output. It was possible that the difference in measurements for the diluted UR variants may result from (i) reduced UR plasmid uptake when forming lipid-DNA complexes for co-transfection with the Gal4-VPR plasmid (which is larger than the DNA-mass-offsetting plasmid Gal4-None) and/or (ii) repression of UR output expression below the autofluorescence threshold.

### Estimation of Cell Concentration by Flow Cytometry

When collecting flow cytometry data, the concentration of cells in a given sample was estimated by the following formula:

$$[ Cells ] ( cells / μL ) = \frac{Event rate ( cells / s )}{Flow rate ( μL / s )}$$

To compute the event rate, the number of cells were estimated (i.e. events passing morphological gating) per second in each sample. The length of time between the measurements of individual cells in flow cytometry approximately follows an exponential distribution. An exponential distribution was fit using the MATLAB function ‘fitdist()’ (https://www.mathworks.com/help/stats/fitdist.html) to the differences between time-stamps of collected cells. Before fitting, inter-cell times larger than the 99.9th percentile was removed to prevent biasing by large outliers. The characteristic parameter of the exponential distribution (λ) is the inverse of the average time between events. Thus, the event rate is given by Embedded Image (i.e. the mean of the exponential distribution).

To ensure a known and controlled flow rate, any sample for which the concentration was measured was collected via the HTS attached to the flow cytometer. The flow rate of the HTS was can be set through the FACSDiva Software (BD) controlling the instrument. The flow rate of each sample was recorded and input into the calculation.

### RT-qPCR

Transfections for qPCR were conducted in 24-well plates (Costar). RNA was collected 48 hours after transfection with the RNeasy Mini kit (Qiagen). Reverse-transcription was performed using the Superscript III kit (Invitrogen) following the manufacturer's recommendations. Real-time qPCR was performed using the KAPA SYBR FAST qPCR 2× master mix (Kapa Biosystems) on a Mastercycler ep Realplex (Eppendorf) following the manufacturer's recommended protocol. Primers for the CMV-driven output (mKate) targeted the coding sequence.

### Primers:

mKate (CMV: Output) forward:
(SEQ ID NO: 2)
GGTGTCTAAGGGCGAAGAGC
mKate (CMV: Output) reverse:
(SEQ ID NO: 3)
GCTGGTAGCCAGGATGTCGA
18S forward:
(SEQ ID NO: 4)
GTAACCCGTTGAACCCCATT
18S reverse:
(SEQ ID NO: 5)
CCATCCAATCGGTAGTAGCG

### Model Fitting

Where possible, fluorescent reporters were used to estimate the concentration of a molecular species for the purpose of model fitting. For fitting the Gal4 activator dose response curves (both activation and competition) in FIGS. 1A-1F, a fluorescent marker co-titrated with the Gal4 activators was used to estimate the amount of Gal4 delivered per cell. The Gal4 marker correlated with the DNA dosage with an R2 value of 0.86 or better for each experimental repeat. However, the sensitivity of activation to Gal4 levels made the measurements as a function of Gal4 DNA dosage relatively noisy between experimental repeats. Thus, the marker levels could more accurately estimate the amount of Gal4 expressed in the median cell than the DNA dosages.

For fitting both the resource competition and iFFL models, the MATLAB function ‘lsqcurvefit()’ (mathworks.com/help/optim/ug/lsqcurvefit.html) was used, which minimizes the sum of the squares of the residuals between the model and the data. As the function input values the level of either the Gal4 TA (in the case of resource competition—as measured by Gal4 Marker) or the transfection marker (in the case of the iFFL) was used. For fitting the Gal4 TA dose-response data, the residuals were computed between the median CMV: Output1 or UAS: Output2 levels and function outputs directly. In addition, all median values computed from different experimental repeats were pooled together before fitting. For fitting iFFL and UR models, the residuals were computed between the log10- and biexponentially-transformed levels of the output protein of interest and the log10- and biexponentially-transformed function outputs, respectively. In experiments with the hEFla iFFL being tested only in HEK-293FT cells, the entire morphologically-gated population of cells was used for fitting. In hEF1a iFFL experiments containing multiple cell types, to prevent the model from over-fitting the untransfected population in more difficult-to-transfect cells, the cells in each sample were analytically binned into half-log-decade-width bins based on the transfection marker, and an equivalent number of cells from each bin were extracted, combined, and used for fitting. In samples with the CMVi iFFL, the relatively high expression of the CMVi promoter compared to the hEF1a promoter (which is used as a transfection marker and proxy for DNA/resource input level z) in most cell lines imposes non-linearity in the transfection marker vs output curve at low plasmid DNA copy numbers per cell. This non-linearity led us to gate cells positive for either the iFFL output or the transfection marker for fitting. For the resource competition models, all parameters for all Gal4 TAs were fit simultaneously using a custom function, ‘lsqmultifit( )’, that was created based on ‘nlinmultifit()’ on the MATLAB file exchange (mathworks.com/matlabcentral/fileexchange/40613-multiple-curve-fitting-with-common-parameters-using-nlinfit).

Goodness of fit was measured by computing the normalized root-mean-square error CV(RMSE). CV(RMSE) was computed with the following equation:

$$CV ( RMSE ) = \frac{\sqrt{\frac{1}{\overline{y}} {\sum }_{i} {( y ( {x}_{i} ) - f ( {x}_{i} ) )}^{2}}}{\overline{y}}$$

Where y(xi) is the value of the data at the input value xi, y̅ is the mean of y for all values of x, and f(xi) is the function output at input value xi.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An engineered incoherent feed forward loop, comprising:
(i) a first transcription unit comprising a first promoter operably linked to a nucleic acid molecule encoding an endoribonuclease; and
(ii) a second transcription unit comprising a second promoter operably linked to a nucleic acid molecule encoding an output molecule, and an endoribonuclease target site located within the 5′ untranslated region (UTR) of the nucleic acid molecule encoding the output molecule,
wherein the endoribonuclease is capable of cleaving the endoribonuclease target site on an RNA transcript expressed by the second transcription unit.

2. The engineered incoherent feed forward loop of claim 1, wherein the first promoter and the second promoter are identical.

3. The engineered incoherent feed forward loop of claim 2, wherein the first promoter and the second promoter share the same transcriptional resources.

4. The engineered incoherent feed forward loop of claim 1, wherein the first promoter and the second promoter are not identical.

5. The engineered incoherent feed forward loop of claim 4, wherein the first promoter is at least 80% identical to the second promoter.

6. The engineered incoherent feed forward loop of claim 1, wherein the endoribonuclease is a CRISPR-associated endoribonuclease.

7. The engineered incoherent feed forward loop of claim 6, wherein the CRISPR-associated endoribonuclease is an endoribonuclease from the Cas6 or Cas13 family optionally wherein the CRISPR-associated endoribonuclease is CasE, Cas6, Csy4, Cse3, PspCas13b, RanCas13b, PguCas13b, or RfxCas13d.

8. (canceled)

9. The engineered incoherent feed forward loop of claim 1, wherein the first transcription unit further comprises at least one upstream open reading frame (uORF) located within the 5′UTR of the nucleotide sequence encoding the endoribonuclease, optionally wherein the uORF comprises a nucleotide sequence of ACCATGGGTTGA (SEQ ID NO: 1), optionally wherein the first transcription unit comprises 1, 2, 3, 4, 5, 6, 7, 8, 9, 10, 11, or 12 uORFs.

10-11. (canceled)

12. The engineered incoherent feed forward loop of any claim 1, wherein the first transcription unit and the second transcription unit are present on the same nucleic acid or on different nucleic acids.

13. The engineered incoherent feed forward loop of claim 12, wherein the first transcription unit and the second transcription unit are present on the same vector or on different vectors.

14. The engineered incoherent feed forward loop of claim 1, wherein the first promoter and/or the second promoter are constitutive promoters, inducible promoters, or tissue specific promoters.

15. A cell comprising the engineered incoherent feed forward loop of claim 1.

16. A composition comprising the engineered incoherent feed forward loop of claim 1.

17. The composition of claim 16, further comprising a pharmaceutically acceptable carrier.

18. A method for delivering an output molecule to a subject in need thereof, the method comprising:
delivering to the subject the engineered incoherent feed forward loop of claim 1.

19. A method for delivering an output molecule to a cell in need thereof, the method comprising:
contacting the cell with the engineered incoherent feed forward loop of claim 1.

20. A method for maintaining expression level of an output molecule to transcriptional disturbance in a subject in need thereof, the method comprising:
delivering to the subject the engineered incoherent feed forward loop of claim 1.

21. The method of claim 18, wherein the first transcription unit and the second transcription unit are delivered on the same nucleic acid or vector.

22. (canceled)

23. The method of claim 18, wherein the first transcription unit and the second transcription unit are delivered on different nucleic acids or different vectors.

24. (canceled)

25. The method of claim 23, wherein the ratio between the first transcription unit and the second transcription unit is proportional.
